(19) United States
(12) Patent Application Publication
(10) Pub. No.: US 2024/0166636 A1
(43) Pub. Date: May 23, 2024

(54) CRYSTALLINE COMPOUNDS AND METHODS OF MAKING THE SAME

(71) Applicant: Epizyme, Inc., Cambridge, MA (US)

(72) Inventors: Marinus Jacobus VERWIJS, Sudbury, MA (US); David Jon AM ENDE, East Lyme, CT (US); Stephen Richard ANDERSON, Stonington, CT (US); Andrew Paul George BEEVERS, Durham (GB); Mark Kenneth BREAULT, Deep River, CT (US); Stephen Richard TUDHOPE, Sunderland (GB); Jamie Ross WOLSTENHULME, Cramlington (GB)

(21) Appl. No.: 18/471,734

(22) Filed: Sep. 21, 2023

Related U.S. Application Data
(63) Continuation of application No. 17/220,245, filed on Apr. 1, 2021, now abandoned.
Application No. 17/220,245 is a continuation of application No. PCT/US2020/066176, filed on Dec. 18, 2020.
(60) Provisional application No. 62/951,842, filed on Dec. 20, 2019.

(51) Int. Cl. C07D 405/12; A61K 9/14; A61K 47/12; A61K 47/26; A61K 47/36; A61K 47/38

(52) U.S. Cl. CPC C07D 405/12; A61K 9/14; A61K 47/12; A61K 47/26; A61K 47/36; A61K 47/38; C07B 2200/13

(57) ABSTRACT

Provided herein are methods for making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide and related products, compositions and treatment methods.

### RELATED APPLICATIONS

This application is a continuation of International Application No. PCT/US2020/066176, filed Dec. 18, 2020, which claims priority to, and the benefit of, U.S. Provisional Application No. 62/951,842, filed Dec. 20, 2019, the contents of each of which are incorporated herein by reference in their entireties.

### TECHNICAL FIELD

This disclosure relates to methods of making crystalline forms of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide, and related products, compositions and treatment methods.

### BACKGROUND

EZH2, a histone methyltransferase, has been associated with various kinds of cancers. Specifically, mutations and/or overactivity of EZH2 are found in a range of cancers, such as lymphomas, leukemias and breast cancer. Moreover it is often advantageous to administer drug products in the form of a salt, for example to aid dissolution or absorption into the body of a patient. In addition, in some cases, certain crystalline forms of pharmaceutical salts are more advantageous than other crystalline forms or amorphous forms.

In the case of crystalline salt drug products, the integrity of the crystal structure, or crystal habit, purity, particle size, and uniformity in the product material (e.g. in the particle size distribution) of a crystalline drug product, as well as efficiency in manufacturing, are important considerations in the crystallization process, and are often difficult to achieve. Hence, there is an ongoing need for new and improved methods of making crystalline forms of EZH2 inhibitors for use in treatment of cancer and other diseases.

### SUMMARY OF THE DISCLOSURE

Provided herein are methods of making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

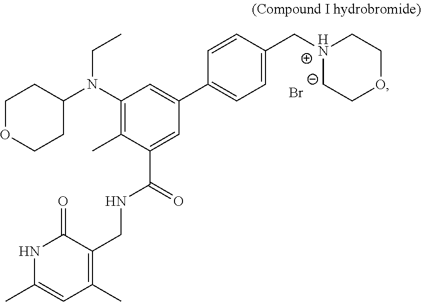

comprising:
step a) mixing Compound I hydrobromide, ethanol, and water, wherein the vol/vol ratio of ethanol:water is from about 92:8 to about 87:13, to form a first mixture.

In some embodiments, provided herein is a method of making a crystalline form of Compound I hydrobromide comprising:
step a) mixing Compound I hydrobromide, ethanol, and water, wherein the vol/vol ratio of ethanol:water is from about 92:8 to about 87:13, to form a first mixture; and
step b) adding a seed to the first mixture to form a second mixture.

In some embodiments, provided herein is a method of making a crystalline form of Compound I hydrobromide comprising:
step a) mixing Compound I hydrobromide, ethanol, and water, wherein the vol/vol ratio of ethanol:water is about 91:9, to form a first mixture; and
step b) adding a seed to the first mixture to form a second mixture.

In some embodiments, provided herein is a method of making a crystalline form of Compound I hydrobromide comprising:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a method of making a crystalline form of Compound I hydrobromide comprising:
step a′) mixing Compound I hydrobromide, ethanol, and water to form a first mixture; and
step b) adding a seed to the first mixture to form a second mixture, wherein step b) is after step a′).

In some embodiments, provided herein is a method of making a crystalline form of Compound I hydrobromide comprising:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a method of making a crystalline form of Compound I hydrobromide consisting essentially of:
step a) mixing Compound I hydrobromide, ethanol, and water, wherein the vol/vol ratio of ethanol and water is from about 92:8 to about 87:13, to form a first mixture; and
step b) adding a seed to the first mixture to form a second mixture; wherein step b) is after step a).

In some embodiments, provided herein is a method of making a crystalline form of Compound I hydrobromide consisting essentially of:
step a) mixing Compound I hydrobromide, ethanol, and water, wherein the vol/vol ratio of ethanol and water is from about 92:8 to about 87:13, to form a first mixture;
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein is a method of making a crystalline form of Compound I hydrobromide consisting essentially of:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a method of making a crystalline form of Compound I hydrobromide consisting essentially of:
step a′) mixing Compound I hydrobromide, ethanol, and water to form a first mixture; and
step b) adding a seed to the first mixture to form a second mixture; wherein step b) is after step a′).

In some embodiments, provided herein is a method of making a crystalline form of Compound I hydrobromide consisting essentially of:
step a′) mixing Compound I hydrobromide, ethanol, and water to form a first mixture; step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein is a method of making a crystalline form of Compound I hydrobromide consisting essentially of:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a method of making N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

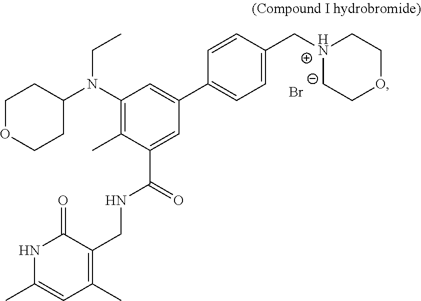

wherein the method comprises:
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), ethanol and toluene to form mixture A; and
step 2) adding hydrobromic acid to mixture A to form a mixture B, wherein Compound I hydrobromide is formed.

In some embodiments, provided herein are particles of a crystalline form of Compound I hydrobromide, wherein the D90 particle size of the particles is from about 15 μm to about 50 μm, and wherein the crystalline form is prepared by a method of the disclosure.

In some embodiments, provided herein is a crystalline form of Compound I hydrobromide.

In some embodiments, provided herein is a plurality of microparticles of a crystalline form of Compound I hydrobromide.

In some embodiments, provided herein is a crystalline form of Compound I hydrobromide, prepared by a method of the disclosure.

In some embodiments, provided herein is a crystalline form of Compound I hydrobromide, wherein the crystalline form forms particles having a D90 particle size of from about 15 μm to about 50 μm.

In some embodiments, provided herein is a solid pharmaceutical composition comprising particles of a crystalline form of Compound I hydrobromide and one or more pharmaceutically acceptable excipients, wherein the crystalline form of Compound I hydrobromide is prepared by a method of the disclosure.

In some embodiments, provided herein is a solid pharmaceutical composition comprising particles of a crystalline form of Compound I hydrobromide and one or more pharmaceutically acceptable excipients, wherein the D90 particle size of the particles is from about 15 μm to about 50 μm, and wherein the crystalline form is prepared by a method of the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### BRIEF DESCRIPTION OF THE DRAWINGS

FIG. 1 is a schematic illustration of a method of the disclosure.

FIG. 2 depicts the differential scanning calorimetry thermogram of a Polymorph A.

FIG. 3 depicts an XRPD diffractogram of a Polymorph A.

### DETAILED DESCRIPTION OF THE DISCLOSURE

### Preparation of Compound I Hydrobromide

Provided herein are methods of making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

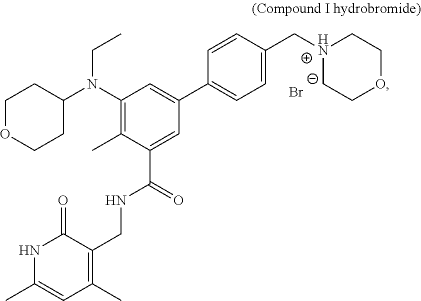

comprising:
step a) mixing Compound I hydrobromide, ethanol, and water, wherein the vol/vol ratio of ethanol:water is from about 92:8 to about 87:13, to form a first mixture.

Also provided herein are methods of making a crystalline form of Compound I hydrobromide comprising: step a′) mixing Compound I hydrobromide, ethanol, and water, to form a first mixture.

Also provided herein are methods of making a crystalline form of Compound I hydrobromide comprising:
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), toluene, and ethanol to form a mixture A; and after step 1):
step 2) adding hydrobromic acid to mixture A to form a mixture B, wherein Compound I hydrobromide is formed.

In some embodiments, the method further comprises after step 2): step 3) adding a seed to mixture B to form mixture C.

In some embodiments, the method further comprises after step 3):
step 4) adding an anti-solvent to mixture C to form mixture D; and after step 4):
step 5) isolating crude Compound I hydrobromide from mixture D.

In some embodiments, the method further comprises after step 5):
step a) mixing Compound I hydrobromide, ethanol, and water, wherein the vol/vol ratio of ethanol:water is from about 92:8 to about 87:13, to form a first mixture.

In some embodiments, the method further comprises after step a): step b) adding a seed to the first mixture to form a second mixture.

In some embodiments, the method further comprises after step 5):
step a) mixing Compound I hydrobromide and a third solvent to form a first mixture; and after step a):
step b) adding a seed to the first mixture to form a second mixture.

In some embodiments, the method of the disclosure further comprises step a-1) heating the first mixture, wherein step a-1) is after step a) or step a′). In some embodiments, the method further comprises step a-2) cooling the first mixture wherein step a-2) is after step a) or step a′), and wherein step a-2) is after step a-1) if step a-1) is present. In some embodiments, the method of the disclosure further comprises after step a): step b) adding a seed to the first mixture to form a second mixture. In some embodiments, the method further comprises step b-1) stirring the second mixture, wherein step b-1) is after step b). In some embodiments, the method further comprises step b-2) cooling the second mixture, wherein step b-2) is after step b), and wherein step b-2) is after step b-1) if step b-1) is present. In some embodiments, the method further comprises step b-3) stirring the second mixture, wherein step b-3) is after step b), wherein step b-3) is after step b-1) if step b-1) is present, and wherein step b-3) is after step b-2) if step b-2) is present. In some embodiments, the method further comprises step c) adding an anti-solvent to the second mixture to form a third mixture, wherein step c) is after step b), and wherein step c) is after any of step b-1), step b-2) and step b-3) that are present. In some embodiments, the method further comprises step d) isolating the crystalline form of Compound I hydrobromide from the third mixture, wherein step d) is after step b), and wherein step d) is after any of step b-1), step b-2), step b-3), and step c) that are present. In some embodiments, the method further comprises step c-1) heating the third mixture, wherein step c-1) is after step c). In some embodiments, the method further comprises step c-2) stirring the third mixture, wherein step c-2) is after step c), and wherein step c-2) is after step c-1) if step c-1) is present. In some embodiments, the method further comprises step c-3) cooling the third mixture, wherein step c-3) is after step c), and wherein step c-3) is after any of step c-1) and step c-2) that are present. In some embodiments, the method further comprises step c-4) stirring the third mixture, wherein step c-4) is after step c), and wherein step c-4) is after any of step c-1), step c-2), and step c-3) that are present. In some embodiments, the method further comprises any combination of any number of steps selected from step a-1), step a-2), step b), step b-1), step b-2), step b-3), step c), and step d).

In some embodiments, the vol/vol ratio of ethanol:water in step a) is from about 92:8 to about 87:13.

In some embodiments the vol/vol ratio of ethanol:water in step a) is about 92:8, about 91.5:8.5, about 91:9, about 90.5:9.5, about 90:10, about 89.5:10.5, about 89:11, about 88.5:11.5, about 88:12, or about 87.5:12.5.

In some embodiments, the vol/vol ratio of ethanol:water in step a) is about 91.3:8.7, about 91.2:8.8, about 91.1:8.9, about 91.0:9.0, about 90.9:9.1, about 90.8:9.2, or about 90.7:9.3.

In some embodiments, the vol/vol ratio of ethanol:water in step a′) is from about 91.5:8.5 to about 87.5:12.5. In some embodiments, the vol/vol ratio of ethanol:water in step a′) is from about 92:8 to about 87:13.

In some embodiments the vol/vol ratio of ethanol:water in step a′) is about 92:8, about 91.5:8.5, about 91:9, about 90.5:9.5, about 90:10, about 89.5:10.5, about 89:11, about 88.5:11.5, about 88:12, or about 87.5:12.5.

In some embodiments, the vol/vol ratio of ethanol:water in step a′) is about 91.3:8.7, about 91.2:8.8, about 91.1:8.9, about 91.0:9.0, about 90.9:9.1, about 90.8:9.2, or about 90.7:9.3.

In some embodiments, in step a-1) the first mixture is heated to a temperature of from about 70° C. to about 75° C. In some embodiments in step a-1) the first mixture is heated to a temperature of about 70° C., about 71° C., about 72° C., about 73° C., about 74° C., or about 75° C.

In some embodiments, in step a-2) the first mixture is cooled to a temperature of from about 45° C. to about 55° C. In some embodiments, in step a-2) the first mixture is cooled to a temperature of from about 50° C. to about 55° C. In some embodiments, in step a-2) the first mixture is cooled to a temperature of about 45° C., about 46° C., about 47° C., about 48° C., about 49° C., about 50° C., about 51° C., about 52° C., about 53° C., about 54° C., or about 55° C.

In some embodiments, in step b) the amount of seed in the second mixture is from about 1.0 wt. % to about 3.0 wt. %. In some embodiments, in step b) the amount of seed in the second mixture is about 1.0 wt. %, about 1.5 wt. %, about 2.0 wt. %, about 2.5 wt. %, or about 3.0 wt. %.

In some embodiments, in step b) the amount of seed in the second mixture is from about 1.96 wt. % to about 2.04 wt. %. In some embodiments, in step b) the amount of seed in the second mixture is about 1.96 wt. %, about 1.97 wt. %, about 1.98 wt. %, about 2.00 wt. %, about 2.01 wt. %, about 2.02 wt. %, about 2.03 wt. %, or about 2.04 wt. %.

In some embodiments, in step b) the D90 particle size of the seed is 6 μm or less. In some embodiments in step b) the D90 particle size of the seed is 5 μm or less. In some embodiments in step b) the D90 particle size of the seed is from about 4 μm to about 6 μm.

In some embodiments, in step b) the D90 particle size of the seed is about 3 μm, about 4 μm, about 5 μm, or about 6 μm.

In some embodiments, the seed in step b) is Compound I hydrobromide. In some embodiments, the seed in step b) is amorphous Compound I hydrobromide. In some embodiments, the seed in step b) is a crystalline form of Compound I hydrobromide. In some embodiments, the seed in step b) is Polymorph A of Compound I hydrobromide. In some embodiments, the seed in step b) exhibits an X-ray powder diffraction pattern having one or two characteristic peaks expressed in degrees 2-theta, selected from the group consisting of 17.5+/−0.3 and 22.0+/−0.3.

In some embodiments, in step b-1) the second mixture is stirred for at least 2 h. In some embodiments, in step b-1) the second mixture is stirred for at least 6 h. In some embodiments in step b-1) the second mixture is stirred for from about 6 h to about 12 h. In some embodiments in step b-1) the second mixture is stirred for about 6 h, about 7 h, about 8 h, about 9 h, about 10 h, about 11 h, or about 12 h.

In some embodiments, in step b-1) the second mixture is stirred at a temperature of from about 45° C. to about 55° C. In some embodiments, in step b-1) the second mixture is stirred at a temperature of from about 50° C. to about 55° C. In some embodiments, in step b-1) the second mixture is stirred at a temperature of about 45° C., about 46° C., about 47° C., about 48° C., about 49° C., about 50° C., about 51° C., about 52° C., about 53° C., about 54° C., or about 55° C.

In some embodiments, in step b-2) the second mixture is cooled at a cooling rate of from about 2° C./h to about 9° C./h. In some embodiments, in step b-2) the second mixture is cooled at a cooling rate of from about 2.5° C./h to about 8.5° C./h. In some embodiments, in step b-2) the second mixture is cooled at a cooling rate of from about 3° C./h to about 8° C./h. In some embodiments, in step b-2) the second mixture is cooled at a cooling rate of about 2° C./h, about 3° C./h, about 4° C./h, about 5° C./h, about 6° C./h, about 7° C./h, about 8° C./h, or about 9° C./h. In some embodiments, in step b-2) the second mixture is cooled at a cooling rate of 3° C./h.

In some embodiments, in step b-2) the second mixture is cooled to a temperature of from about 18° C. to about 30° C. In some embodiments, in step b-2) the second mixture is cooled to a temperature of from about 20° C. to about 25° C. In some embodiments, in step b-2) the second mixture is cooled to a temperature of about 18° C., about 19° C., about 20° C., about 21° C., about 22° C., about 23° C., about 24° C., about 25° C., about 26° C., about 27° C., about 28° C., about 29° C., or about 30° C. In some embodiments, the second mixture is cooled to a temperature of 22° C.

In some embodiments, in step b-3) the second mixture is stirred for about 3 h to about 15 h. In some embodiments, in step b-2) the second mixture is stirred for at least about 3 h, at least about 4 h, at least about 5 h, at least about 5 h, at least about 7 h, at least about 8 h, at least about 9 h, at least about 10 h, at least about 11 h, at least about 12 h, at least about 13 h, at least about 14 h, or at least about 15 h. In some embodiments, in step b-2) the second mixture is stirred for about 3 h, about 4 h, about 5 h, about 6 h, about 7 h, about 8 h, about 9 h, about 10 h, about 11 h, about 12 h, about 13 h, about 14 h, or about 15 h. In some embodiments, in step b-2) the second mixture is stirred for ≥16 h.

In some embodiments, in step c) the anti-solvent is added over a time period of from about 1 h to about 5 h. In some embodiments, in step c) the anti-solvent is added over a time period of from about 3 h to about 5 h. In some embodiments, in step c) the anti-solvent is added over a time period of about 1 h, about 2 h, about 3 h, about 4 h, or about 5 h.

In some embodiments, in step c), the entire amount of the anti-solvent is added at once.

In some embodiments, in step c) the anti-solvent is added in amount of from about 5 volumes to about 15 volumes. In some embodiments, in step c) the anti-solvent is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

In some embodiments, in step c) the anti-solvent is added until crystalline particles of crystalline form of Compound I hydrobromide form.

In some embodiments, the anti-solvent in step c) is selected from ethyl acetate, methyl tert-butyl ether, tetrahydrofuran, and acetone. In some embodiments, the anti-solvent in step c) is ethyl acetate.

In some embodiments, in step c) ethyl acetate is added over a time period of from about 1 h to about 5 h. In some embodiments, in step c) ethyl acetate is added over a time period of from about 3 h to about 5 h. In some embodiments, in step c) ethyl acetate is added over a time period of about 1 h, about 2 h, about 3 h, about 4 h, or about 5 h.

In some embodiments, in step c), the entire amount of ethyl acetate is added at once.

In some embodiments, in step c) ethyl acetate is added in amount of from about 5 volumes to about 15 volumes. In some embodiments, in step c) ethyl acetate is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

In some embodiments, in step c) ethyl acetate is added until crystalline particles of Compound I hydrobromide form.

In some embodiments, in step c-1) the third mixture is heated to a temperature of from about 45° C. to about 55° C. In some embodiments, in step c-1) the third mixture is heated to a temperature of from about 47° C. to about 53° C. In some embodiments, in step c-1) the third mixture is heated to a temperature of about 47° C., about 48° C., about 49° C., about 50° C., about 51° C., about 52° C., or about 53° C.

In some embodiments, in step c-2) the third mixture is stirred for at least about 1 h. In some embodiments, in step c-2) the third mixture is stirred for about 1 h, about 2 h, about 3 h, about 4 h, or about 5 h or more.

In some embodiments, in step c-3) the third mixture is cooled to a temperature of from about 10° C. to about 40° C. In some embodiments, in step c-3) the third mixture is cooled to a temperature of from about 10° C. to about 35° C. In some embodiments, in step c-3) the third mixture is cooled to a temperature of from about 18° C. to about 35° C. In some embodiments, in step c-3) the third mixture is cooled to a temperature of from about 10° C. to about 20° C. In some embodiments, in step c-3) the third mixture is cooled to a temperature of from about 13° C. to about 18° C. In some embodiments, in step c-3) the third mixture is cooled to a temperature of about 13° C., about 14° C., about 15° C., about 16° C., about 17° C., or about 18° C. In some embodiments, in step c-3) the third mixture is cooled over the course of about 1 h. In some embodiments, in step c-3) the third mixture is cooled over the course of about 2 h, about 3 h, about 4 h, or about 5 h.

In some embodiments, in step c-4) the third mixture is stirred for at least about 1 h. In some embodiments, in step c-4) the third mixture is stirred for about 1 h, about 2 h, about 3 h, about 4 h, or about 5 h or more.

In some embodiments, in step d) the crystalline form of Compound I hydrobromide is isolated from the third mixture by filtration.

In some embodiments, the method further comprises before step a) or step a′):
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), ethanol and toluene to form mixture A; and
step 2) adding hydrobromic acid to mixture A to form mixture B, wherein Compound I hydrobromide is formed; and wherein step 2) is after step 1).

In some embodiments, the method further comprises before step a) or step a′):
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D; wherein step 4-1) is after step 4); and
step 5) isolating crude Compound I hydrobromide from mixture D; wherein step 5) is after step 4-1).

In some embodiments, the method of the disclosure consists essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a) mixing Compound I hydrobromide, ethanol, and water, wherein the vol/vol ratio of ethanol and water is from about 92:8 to about 87:13, to form a first mixture; wherein step a) is after step 2); and
step b) adding a seed to the first mixture to form a second mixture; wherein step b) is after step a).

In some embodiments, the method of the disclosure consists essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a′) mixing Compound I hydrobromide, ethanol, and water, wherein the vol/vol ratio of ethanol and water is from about 92:8 to about 87:13, to form a first mixture; wherein step a) is after step 2); and
step b) adding a seed to the first mixture to form a second mixture; wherein step b) is after step a′).

In some embodiments, the method of the disclosure consists essentially of:
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D; wherein step 4-1) is after step 4);
step 5) isolating crude Compound I hydrobromide from mixture D; wherein step 5) is after step 4-1);
step a) mixing Compound I hydrobromide, ethanol, and water, wherein the vol/vol ratio of ethanol and water is from about 92:8 to about 87:13, to form a first mixture; wherein step a) is after step 5);
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, the method of the disclosure consists essentially of:
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D; wherein step 4-1) is after step 4);
step 5) isolating crude Compound I hydrobromide from mixture D; wherein step 5) is after step 4-1);
step a′) mixing Compound I hydrobromide, ethanol, and water, wherein the vol/vol ratio of ethanol and water is from about 92:8 to about 87:13, to form a first mixture; wherein step a) is after step 5);
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, in step 1) the vol/vol ratio of ethanol:toluene in mixture A is from about 25:75 to about 45:55. In some embodiments, in step 1) the vol/vol ratio of ethanol:toluene in mixture A is about 25:75, about 30:70, about 35:65, about 40:60, or about 45:55.

In some embodiments, in step 1-1), mixture A is heated to a temperature of from about 40° C. to about 80° C. In some embodiments, in step 1-1), mixture A is heated to a temperature of from about 60° C. to about 70° C. In some embodiments, in step 1-1), mixture A is heated to a temperature of about 40° C., about 45° C., about 50° C., about 55° C., about 60° C., about 65° C., about 70° C., about 75° C., or about 80° C.

In some embodiments, in step 1-2), mixture A is cooled to a temperature of from about 20° C. to about 40° C. In some embodiments, in step 1-2), mixture A is cooled to a temperature of from about 25° C. to about 35° C. In some embodiments, in step 1-2), mixture A is cooled to a temperature of about 20° C., about 25° C., about 30° C., about 35° C., or about 40° C. In some embodiments, in step 1-2), mixture A is cooled to a temperature of 30° C.

In some embodiments, in step 2), hydrobromic acid is added to mixture B at a temperature of from about 10° C. to about 50° C. In some embodiments, in step 2), hydrobromic acid is added to mixture B at a temperature of from about 20° C. to about 40° C. In some embodiments, in step 2), hydrobromic acid is added to mixture B at a temperature of from about 25° C. to about 35° C. In some embodiments, in step 2), hydrobromic acid is added to mixture B at a temperature of about 10° C., about 15° C., 20° C., about 25° C., about 30° C., about 35° C., about 40° C., about 45° C., or about 50° C. In some embodiments, in step 2), hydrobromic acid is added to mixture B at a temperature of 30° C.

In some embodiments, in step 2) hydrobromic acid is added to mixture A in an amount of from about 0.9 mol eq. to about 1.1 mol eq. with respect to Compound I. In some embodiments, in step 2) hydrobromic acid is added to mixture A in an amount of from about 0.95 mol eq. to about 1.05 mol eq. with respect to Compound I. In some embodiments, in step 2) hydrobromic acid is added to mixture A in an amount of from about 0.975 mol eq. to about 0.990 mol eq. with respect to Compound I. In some embodiments, in step 2) hydrobromic acid is added to mixture A in an amount of from about 0.975 mol eq. to about 0.995 mol eq. with respect to Compound I.

In some embodiments, in step 2) hydrobromic acid is added to mixture A in an amount of from about 0.98 mol eq. to about 1.00 mol eq. with respect to Compound I. In some embodiments, in step 2) hydrobromic acid is added to mixture A in an amount of about 0.95 mol eq., about 0.96 mol eq., about 0.97 mol eq., about 0.98 mol eq., about 0.99 mol eq., about 1.00 mol eq., about 1.01 mol eq., about 1.02 mol eq., about 1.03 mol eq., about 1.04 mol eq., or about 1.05 mol eq. with respect to Compound I. In some embodiments, in step 2) hydrobromic acid is added to mixture A in an amount of 0.99 mol eq. with respect to Compound I. In some embodiments, in step 2) hydrobromic acid is added in an amount of 0.985 mol eq. with respect to Compound I.

In some embodiments, in step 3) the amount of seed in mixture B is from about 1.96 wt. % to about 2.04 wt. %. In some embodiments, in step 3) the amount of seed in mixture B is about 1.96 wt. %, about 1.97 wt. %, about 1.98 wt. %, about 2.00 wt. %, about 2.01 wt. %, about 2.02 wt. %, about 2.03 wt. %, or about 2.04 wt. %.

In some embodiments, in step 3) the D90 particle size of the seed is 6 μm or less. In some embodiments, in step 3) the D90 particle size of the seed is 5 μm or less. In some embodiments, in step 3) the D90 particle size of the seed is from about 4 μm to about 6 μm.

In some embodiments, in step 3) the D90 particle size of the seed is about 3 μm, about 4 μm, about 5 μm, or about 6 μm.

In some embodiments, the seed in step 3) is Compound I hydrobromide. In some embodiments, the seed in step 3) is amorphous Compound I hydrobromide. In some embodiments, the seed in step 3) is a crystalline form of Compound I hydrobromide. In some embodiments, the seed in step 3) is Polymorph A of Compound I hydrobromide. In some embodiments, the seed in step 3) exhibits an X-ray powder diffraction pattern having one or two characteristic peaks expressed in degrees 2-theta, selected from the group consisting of 17.5+/−0.3 and 22.0+/−0.3.

In some embodiments, in step 3-1), the mixture is cooled to a temperature of from about 0° C. to about 20° C. In some embodiments, in step 3-1), the mixture is cooled to a temperature of from about 5° C. to about 15° C. In some embodiments, in step 3-1), the mixture is cooled to a temperature of about 5° C., about 6° C., about 7° C., about 8° C., about 9° C., about 10° C., about 11° C., about 12° C., about 13° C., about 14° C., or about 15° C.

In some embodiments, in step 4) the anti-solvent is added over a time period of from about 1 h to about 5 h. In some embodiments, in step 4) the anti-solvent is added over a time period of from about 3 h to about 5 h. In some embodiments, in step 4) the anti-solvent is added over a time period of about 1 h, about 2 h, about 3 h, about 4 h, or about 5 h.

In some embodiments, in step 4), the entire amount of the anti-solvent is added at once.

In some embodiments, in step 4) the anti-solvent is added in amount of from about 5 volumes to about 15 volumes. In some embodiments, in step 4) the anti-solvent is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

In some embodiments, in step 4) the anti-solvent is added until crystalline particles of Compound I hydrobromide form.

In some embodiments, the anti-solvent in step 4) is selected from ethyl acetate, methyl tert-butyl ether, tetrahydrofuran, and acetone. In some embodiments, in step 4) the anti-solvent is ethyl acetate.

In some embodiments, in step 4) ethyl acetate is added over a time period of from about 1 h to about 5 h. In some embodiments, in step 4) ethyl acetate is added over a time period of from about 3 h to about 5 h. In some embodiments, in step 4) ethyl acetate is added over a time period of about 1 h, about 2 h, about 3 h, about 4 h, or about 5 h.

In some embodiments, in step 4), the entire amount of ethyl acetate is added at once.

In some embodiments, in step 4) ethyl acetate is added in amount of from about 5 volumes to about 15 volumes. In some embodiments, in step 4) ethyl acetate is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

In some embodiments, in step 4) ethyl acetate is added until crystalline particles of Compound I hydrobromide form.

In some embodiments, in step 4-1) mixture D is stirred for ≥4 h. In some embodiments, in step 4-1) mixture D is stirred for from about 4 h to about 15 h. In some embodiments, in step 4-1) mixture D is stirred for about 4 h, about 5 h, about 6 h, about 7 h, about 8 h, about 9 h, about 10 h, about 11 h, about 12 h, about 13 h, about 14 h, or about 15 h.

In some embodiments, in step 5) crude Compound I hydrobromide is isolated from mixture D by filtration.

As used herein, the term “about” refers to a recited amount, value, or duration ±10% or less of said amount, value, or duration. In some embodiments, “about” refers to a recited amount, value, or duration ±10%, ±8%, ±6%, ±5%, ±40%, ±20%, ±1%, or ±0.5%. In other embodiments, “about” refers to a recited amount, value, or duration ±10%, ±8%, ±6%, ±5%, ±4%, or ±2%. In other embodiments, “about” refers to a recited amount, value, or duration ±5%. In some embodiments, “about” refers to a listed amount, value, or duration ±2% or ±1%. For example, in some embodiments, when the term “about” is used when reciting a temperature or temperature range, these terms refer to the recited temperature or temperature range ±5° C., ±2° C., or ±1° C. In other embodiments, the term “about” refers to the recited temperature or temperature range ±2° C.

For example, in some embodiments, when the term “about” is used when reciting a duration or duration range, the term refers to the recited duration or duration range ±6 min, ±4 min, or ±2 min. In some embodiments, the term “about” refers to the recited duration or duration range ±5 min.

As set forth in Example 1, in some embodiments, the method of the disclosure provides a robust process for making a crystalline form of Compound I hydrobromide, wherein the crystalline form is Polymorph A. For example, in some embodiments, the method of the disclosure consistently produces Polymorph A. Without wishing to be bound by theory, in some embodiments, using ethanol and water at a specific vol/vol ratio (e.g., from about 92:8 to about 87:13, or from about 91.5:8.5 to about 87.5:12.5, e.g., about 91:9) in step a) results in robust production of Polymorph A.

Furthermore, as set forth in Example 1, in some embodiments, the method of the present disclosure provides a highly pure crystalline form of Compound I hydrobromide. For example, in some embodiments, the method of the disclosure provides a crystalline form of Compound I hydrobromide that is at least 95%, 96%, 97%, 98% or 99% pure. For example, in some embodiments, the method of the disclosure provides a crystalline form of Compound I hydrobromide that is at least 99.5%, 99.6%, 99.7%, 99.8%, or 99.9% pure.

As set forth in Example 1, in some embodiments, the method of the disclosure allows for the addition of HBr in the formation of Compound I hydrobromide at low temperatures (e.g., temperatures of from about 10° C. to about 50° C., from about 20° C. to about 40° C., or from about 25° C. to about 35° C.). Without wishing to be bound by theory, in some embodiments, using toluene and water as a solvent in step 1) of a method of the disclosure provides a homogeneous solution of Compound I at temperatures of from about 10° C. to about 50° C., from about 20° C. to about 40° C., or from about 25° C. to about 35° C., whereas e.g., using ethanol and water as the solvent for Compound I in step 1) may require higher temperatures (e.g., between 65° C.-75° C.). Without wishing to be bound by theory, in some embodiments, this minimizes the occurrence of impurities resulting from degradation processes, e.g., N-dealkylation decomposition impurities. Accordingly, in some embodiments, the method of the disclosure produces a crystalline form of Compound I hydrobromide which does not contain, or does not contain a significant amount of, degradation impurities (e.g., N-dealkylation decomposition impurities). For example, in some embodiments, a crystalline form of Compound I hydrobromide made by a method of the disclosure contains no N-dealkylation decomposition impurities.

Without wishing to be bound by theory, in some embodiments, recrystallization to purge residual toluene in Compound I hydrobromide made by a method of the disclosure resulted in entrapment of other residual solvents (e.g., ethyl acetate, ethanol), within the crystalline particles of Compound I hydrobromide. Without wishing to be bound by theory, in some embodiments, entrapment of residual solvents (e.g., ethyl acetate, ethanol) can lead to high levels of residual solvent that cannot be purged by drying. Without wishing to be bound by theory, in some embodiments, excess presence of Polymorph B of Compound I hydrobromide contributes to entrapment of residual solvent. In some embodiments, the method of the disclosure solves the problem of high levels of residual solvent. In some embodiments, increasing the amount of seed in step b) reduces the residual solvent levels. In some embodiments, decreasing the size of the seed used in step b) decreases residual solvent levels.

As set forth in Example 1, in some embodiments, the method of the disclosure provides a crystalline form of Compound I hydrobromide containing low levels of residual solvents. For example, in some embodiments, the method of the disclosure provides a crystalline form of Compound I hydrobromide containing about 350 ppm or less (e.g. about 300 ppm or less, about 250 ppm or less, about 200 ppm or less, about 150 ppm or less, about 100 ppm or less, or about 50 ppm or less, e.g., about 320 ppm or less) of residual ethanol. In some embodiments, the method of the disclosure provides a crystalline form of Compound I hydrobromide containing less than 100 ppm (e.g., about 80 ppm or less, about 75 ppm or less, about 70 ppm or less, about 65 ppm or less, about 60 ppm or less, about 55 ppm or less, about 50 ppm or less, about 45 ppm or less, about 40 ppm or less, about 35 ppm or less, about 30 ppm or less, about 25 ppm or less, about 20 ppm or less, about 15 ppm or less, or about 10 ppm or less) of residual ethyl acetate. In some embodiments, the method of the disclosure provides a crystalline form of Compound I hydrobromide containing 25 ppm or less (e.g., about 20 ppm or less, about 15 ppm or less, about 10 ppm or less, or about 5 ppm or less) of residual toluene. In some embodiments, the amount of seed used in step b) affects properties of the crystalline particles of Compound I hydrobromide (e.g., particle size or residual solvent levels). In some embodiments, the size of the seed used in step b) affects properties of the crystalline particles of Compound I hydrobromide (e.g. particle size or residual solvent levels). In some embodiments, cooling the reaction mixture after seeding improves the properties of the crystalline particles of Compound I hydrobromide (e.g. particle size or residual solvent levels). In some embodiments, the rate at which the reaction mixture is cooled after seeding impacts properties of the crystalline particles of Compound I hydrobromide (e.g. particle size or residual solvent levels). For example, in some embodiments, the cooling rate in step b-2) has an impact on properties of the crystalline particles of Compound I hydrobromide (e.g. particle size or residual solvent levels). For example, in some embodiments, increasing the cooling rate in step b-2) decreases the size of the resulting particles. For example, in some embodiments, increasing the cooling rate in step b-2) decreases the residual solvent levels.

As further set forth in Example 1, in some embodiments, the method of the disclosure also provides crystalline particles of a crystalline form of Compound I hydrobromide which have a suitable particle size (for example, in some embodiments, the crystalline form of the disclosure has a D90 particle size of from about 15 μm to about 50 μm. For example, in some embodiments, the crystalline form of Compound I hydrobromide has a D90 particle size of about 15 μm, about 20 μm, about 25 μm, about 30 μm, about 35 μm, about 40 μm, about 45 μm, or about 50 μm. For example, in some embodiments, the crystalline form of Compound I hydrobromide has a D90 particle size of about 31 μm). Moreover, in some embodiments, the method of the disclosure produces particles of a crystalline form of Compound I hydrobromide of a narrow size distribution (i.e., a size distribution where a majority of the particles has a diameter close to the mean particle size.

Furthermore, in some embodiments, the method of the disclosure produces a symmetric, unimodal (i.e. single peaked) particle size distribution. In other words, crystalline particles made by a method of the disclosure are devoid of any significant secondary populations (i.e., particle populations with a size distribution not centered within the primary population, which may shift the shape of the overall size distribution).

In some embodiments, the unique combination of the steps described in the disclosure improves the properties of a crystalline form of Compound I hydrobromide made by a method of the disclosure. In some embodiments, the specific order of steps comprised in a method of the disclosure improves the properties of a crystalline form of Compound I hydrobromide made by a method of the disclosure.

Lastly, in some embodiments, the method of the disclosure is suitable for production on a large-scale. For example, in some embodiments, the method of the disclosure can be conducted at lower temperature than previous methods, allowing for easier scale-up.

### Polymorph of Compound I Hydrobromide

Provided herein are crystalline forms of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

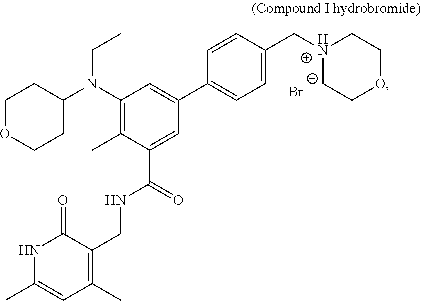

wherein the crystalline form forms particles having a D90 particle size of from about 15 μm to about 50 μm. In some embodiments, provided herein is a crystalline form of Compound I hydrobromide wherein the crystalline form forms particles having a D90 particle size of about 31 μm.
In some embodiments, provided herein is a crystalline form of Compound I hydrobromide made by a method of the disclosure.

In some embodiments, provided is a plurality of microparticles of Compound I hydrobromide, wherein the D90 particle size of the microparticles is from about 15 μm to about 50 μm.

In some embodiments, provided herein is a crystalline form of Compound I hydrobromide made by a method comprising:
step a) mixing Compound I hydrobromide, ethanol, and water, wherein the vol/vol ratio of ethanol:water is from about 92:8 to about 87:13, to form a first mixture; and
step b) adding a seed to the first mixture to form a second mixture.

In some embodiments, provided herein is a crystalline form of Compound I hydrobromide made by a method comprising:
step a) mixing Compound I hydrobromide, ethanol, and water, wherein the vol/vol ratio of ethanol and water is from about 92:8 to about 87:13, to form a first mixture;
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein is a crystalline form of Compound I hydrobromide made by a method comprising:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a crystalline form of Compound I hydrobromide made by a method comprising:
step a′) mixing Compound I hydrobromide, ethanol, and water to form a first mixture; and
step b) adding a seed to the first mixture to form a second mixture, wherein step b) is after step a′).

In some embodiments, provided herein is a crystalline form of Compound I hydrobromide made by a method comprising:
step a′) mixing Compound I hydrobromide, ethanol, and water to form a first mixture; step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein is a crystalline form of Compound I hydrobromide made by a method comprising:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein a crystalline form of Compound I hydrobromide made by a method consisting essentially of:
step a) mixing Compound I hydrobromide, ethanol, and water, wherein the vol/vol ratio of ethanol and water is from about 92:8 to about 87:13, to form a first mixture; and
step b) adding a seed to the first mixture to form a second mixture; wherein step b) is after step a).

In some embodiments, provided herein is a crystalline form of Compound I hydrobromide made by a method consisting essentially of:
step a) mixing Compound I hydrobromide, ethanol, and water, wherein the vol/vol ratio of ethanol and water is from about 92:8 to about 87:13, to form a first mixture;
step b) adding a seed to the first mixture to form a second mixture; wherein step b) is after step a);
step c) adding an anti-solvent to the second mixture to form a third mixture; wherein step c) is after step b); and
step d) isolating the a crystalline form of Compound I hydrobromide from the third mixture; wherein step d) is after step c).

In some embodiments, provided herein is a crystalline form of Compound I hydrobromide made by a method consisting essentially of:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a crystalline form of Compound I hydrobromide made by a method consisting essentially of:
step a′) mixing Compound I hydrobromide, ethanol, and water to form a first mixture; and
step b) adding a seed to the first mixture to form a second mixture; wherein step b) is after step a′).

In some embodiments, provided herein is a crystalline form of Compound I hydrobromide made by a method consisting essentially of:
step a′) mixing Compound I hydrobromide, ethanol, and water to form a first mixture;
step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein is a crystalline form of Compound I hydrobromide made by a method consisting essentially of:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein is a crystalline form of Compound I hydrobromide made by a method comprising:
step 1) mixing Compound I, ethanol, and toluene to form mixture A;
step 2) adding hydrobromic acid to mixture A to form mixture B, wherein Compound I hydrobromide is formed; and wherein step 2) is after step 1).

In some embodiments, provided herein is a crystalline form of Compound I hydrobromide made by a method consisting essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a) mixing Compound I hydrobromide, ethanol, and water, wherein the vol/vol ratio of ethanol and water is from about 92:8 to about 87:13, to form a first mixture; wherein step a) is after step 2); and
step b) adding a seed to the first mixture to form a second mixture; wherein step b) is after step a).

In some embodiments, provided herein is a crystalline form of Compound I hydrobromide made by a method consisting essentially of:
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D; wherein step 4-1) is after step 4);
step 5) isolating crude Compound I hydrobromide from mixture D; wherein step 5) is after step 4-1);
step a) mixing Compound I hydrobromide, ethanol, and water, wherein the vol/vol ratio of ethanol and water is from about 92:8 to about 87:13, to form a first mixture; wherein step a) is after step 5);
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is prepared by a method comprising:
step a) mixing Compound I hydrobromide, ethanol, and water, wherein the vol/vol ratio of ethanol:water is from about 92:8 to about 87:13, to form a first mixture; and
step b) adding a seed to the first mixture to form a second mixture.

In some embodiments, provided herein are particles of a crystalline form of Compound I hydrobromide, wherein the 90% cumulative particle diameter of the particles is from about 15 μm to about 50 μm, and wherein the crystalline form is made by a method comprising:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method comprising:
step a′) mixing Compound I hydrobromide, ethanol, and water to form a first mixture; and
step b) adding a seed to the first mixture to form a second mixture, wherein step b) is after step a′).

In some embodiments, provided herein are particles of a crystalline form of Compound I hydrobromide, wherein the 90% cumulative particle diameter of the particles is from about 15 μm to about 50 μm, and wherein the crystalline form is made by a method comprising:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a) mixing Compound I hydrobromide, ethanol, and water, wherein the vol/vol ratio of ethanol and water is from about 92:8 to about 87:13, to form a first mixture; and
step b) adding a seed to the first mixture to form a second mixture; wherein step b) is after step a).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a) mixing Compound I hydrobromide, ethanol, and water, wherein the vol/vol ratio of ethanol and water is from about 92:8 to about 87:13, to form a first mixture;
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a) mixing Compound I hydrobromide, ethanol, and water, wherein the vol/vol ratio of ethanol and water is from about 92:8 to about 87:13, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a′) mixing Compound I hydrobromide, ethanol, and water to form a first mixture; and
step b) adding a seed to the first mixture to form a second mixture; wherein step b) is after step a′).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a′) mixing Compound I hydrobromide, ethanol, and water to form a first mixture;
step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a) mixing Compound I hydrobromide, ethanol, and water, wherein the vol/vol ratio of ethanol and water is from about 92:8 to about 87:13, to form a first mixture; wherein step a) is after step 2); and
step b) adding a seed to the first mixture to form a second mixture; wherein step b) is after step a).

In some embodiments, provided herein are particles of a crystalline form of Compound I hydrobromide, wherein the 90% cumulative particle diameter of the particles is from about 15 μm to about 50 μm, and wherein the crystalline form is made by a method consisting essentially of:
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D; wherein step 4-1) is after step 4);
step 5) isolating crude Compound I hydrobromide from mixture D; wherein step 5) is after step 4-1);
step a) mixing Compound I hydrobromide, ethanol, and water, wherein the vol/vol ratio of ethanol and water is from about 92:8 to about 87:13, to form a first mixture; wherein step a) is after step 5);
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).

As used herein, “Compound I” refers to N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide. The hydrobromide of Compound I can be used to inhibit the histone methyltransferase activity of EZH2, either in a subject or in vitro. The hydrobromide of Compound I can also be used to treat cancer in a subject in need thereof.

Compound I can be protonated at one or more of its basic sites, such as the morpholine, disubstituted aniline, and/or pyridone moieties. Hydrobromide salts of Compound I can occur as a monohydrobromide, dihydrobromide, or trihydrobromide. As used herein, “Compound I hydrobromide” refers to the monohydrobromide of Compound I. When the compound is the monohydrobromide, the compound may be protonated at any basic site. In a non-limiting embodiment, Compound I is protonated at the nitrogen of the morpholino substituent, providing a monohydrobromide of Compound I having the following structure:

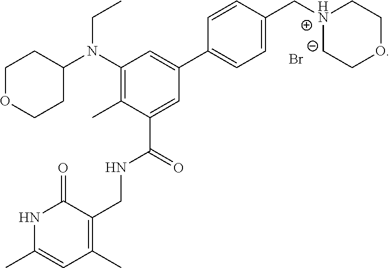

This particular monohydrobromide can be referred to as “4-((3′-(((4,6-dimethyl-2-oxo-1,2-dihydropyridin-3-yl)methyl)carbamoyl)-5′-(ethyl(tetrahydro-2H-pyran-4-yl)amino)-4′-methyl-[1,1′-biphenyl]-4-yl)methyl)morpholin-4-ium bromide.”

The hydrobromide of Compound I has a number of advantageous physical properties over its free base form, as well as other salts of the free base. In particular, the hydrobromide of Compound I has low hygroscopicity compared to other salt forms of Compound I. For a compound to be effective in therapy, it is generally required that the compound be minimally hygroscopic. Drug forms that are highly hygroscopic may be unstable, as the drug form's dissolution rate may change as it is stored in settings with varying humidity. Also, hygroscopicity can impact large-scale handling and manufacturing of a compound, as it can be difficult to determine the true weight of a hygroscopic active agent when preparing a pharmaceutical composition comprising that agent. The hydrobromide of Compound I has a low hygroscopicity compared to other salt forms of Compound I. As such, it can be stored over appreciable periods, and will not suffer from detrimental changes in, for example, solubility, density, or even chemical composition.

In addition to the above advantages, the hydrobromide of Compound I can be produced in a highly crystalline form, which is useful in the preparation of pharmaceutical compositions, and will improve general handling, manipulation, and storage of the drug compound. In a preferred embodiment, the crystalline form of the hydrobromide of Compound I is in a form referred to as “Polymorph A” or “Form A.”

The ability of a substance to exist in more than one crystal form is defined as polymorphism; the different crystal forms of a particular substance are referred to as “polymorphs.” In general, polymorphism is affected by the ability of a molecule of a substance to change its conformation or to form different intermolecular or intra-molecular interactions, particularly hydrogen bonds, which is reflected in different atom arrangements in the crystal lattices of different polymorphs. In contrast, the overall external form of a substance is known as “morphology,” which refers to the external shape of the crystal and the planes present, without reference to the internal structure. Examples of a substances morphology includes but is not limited to cubes, platelets, and spheres. Crystals can display different morphology based on different conditions, such as, for example, growth rate, stirring, and the presence of impurities. Crystal morphology is a quality trait that plays a role in many downstream drug product processes. Morphology may affect particles' properties such as flow-ability, filtration, drying, and ultimately tablet dissolution. In some embodiments, the morphology of the crystalline form of the disclosure is that of cubes. In some embodiments, the morphology of the crystalline form of the disclosure is that of platelets. In some embodiments, the morphology of the crystalline form of the disclosure is that of spheres.

The different crystalline forms of a substance may possess different energies of the crystal lattice and, thus, in solid state they can show different physical properties such as form, density, melting point, color, stability, solubility, dissolution rate, etc., which can, in turn, affect the stability, dissolution rate and/or bioavailability of a given polymorph and its suitability for use as a pharmaceutical and in pharmaceutical compositions.

Without wishing to be bound by theory, polymorph forms exhibiting compact crystal shapes possess advantages in terms of ease of filtration and ease of flow. Polymorph A exhibits a compact crystal shape that therefore possesses these advantages.

Moreover, as shown in the table below, Polymorph A has a higher dissolution rate than other polymorphs of Compound I, Compound I hydrobromide or the bis-hydrobromide of Compound I.

| | Intrinsic Dissolution Rate | |
|---|---|---|
| Polymorph | (IDR) (mg/min/cm²) | % Rate relative to From A |
| A | 7.6 | 100 |
| B | 6.4 | 85 |
| M | 5.5 | 72 |
| 1 | 4.4 | 59 |
| 2 | 6.4 | 85 |
| Bis-HBr | 10.3 | 137 |

In some embodiments, Polymorph A is identifiable on the basis of characteristic peaks in an X-ray powder diffraction analysis. X-ray powder diffraction, also referred to as XRPD, is a scientific technique using X-ray, neutron, or electron diffraction on powder, microcrystalline, or other solid materials for structural characterization of the materials. In some embodiments, the Polymorph A exhibits an X-ray powder diffraction pattern having one or two characteristic peaks expressed in degrees 2-theta, selected from the group consisting of 17.5+/−0.3, and 22.0+/−0.3.

In some embodiments, Polymorph A exhibits an X-ray powder diffraction pattern having one or more characteristic peaks expressed in degrees 2-theta, selected from the group consisting of 3.9+/−0.3, 17.5+/−0.3, and 22.0+/−0.3. In some embodiments, the crystalline form exhibits an X-ray powder diffraction pattern having characteristic peaks expressed in degrees 2-theta at 3.9+/−0.3, 17.5+/−0.3, and 22.0+/−0.3.

In some embodiments, Polymorph A exhibits an X-ray powder diffraction pattern having at least 5 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 6 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 7 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 8 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 9 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 10 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.

In some embodiments, Polymorph A exhibits an X-ray powder diffraction pattern having characteristic peaks expressed in degrees 2-theta at 3.9+/−0.3, 14.3+/−0.3, 18.7+/−0.3, 23.3+/−0.3, and 23.6+/−0.3.

In some embodiments, Polymorph A exhibits an X-ray powder diffraction pattern having characteristic peaks expressed in degrees 2-theta at 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.

In some embodiments, Polymorph A exhibits an X-ray powder diffraction pattern having one or more characteristic peaks expressed in degrees 2-theta, selected from the group consisting of 17.5+/−0.3, and 22.0+/−0.3. In some embodiments, the crystalline form exhibits an X-ray powder diffraction pattern having characteristic peaks expressed in degrees 2-theta at 17.5+/−0.3, and 22.0+/−0.3.

In some embodiments, Polymorph A exhibits an X-ray powder diffraction pattern having at least 5 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 6 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 7 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 8 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 9 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3. In some embodiments, Polymorph A exhibits an X-ray powder diffraction pattern having at least 10 characteristic peaks expressed in degrees 2-theta, selected from the group consisting of 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.

In some embodiments, Polymorph A exhibits an X-ray powder diffraction pattern having characteristic peaks expressed in degrees 2-theta at 14.3+/−0.3, 18.7+/−0.3, 23.3+/−0.3, and 23.6+/−0.3.

In some embodiments, Polymorph A exhibits an X-ray powder diffraction pattern having characteristic peaks expressed in degrees 2-theta at 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.

In some embodiments, Polymorph A exhibits an X-ray powder diffraction pattern substantially in accordance with the 2-theta values listed in Table 1.

Compositions comprising Polymorph A can be identified by comparison of the compositions' X-ray powder diffraction patterns to an X-ray powder diffraction pattern of Polymorph A. It will be appreciated that pharmaceutical compositions comprising Polymorph A may exhibit non-identical X-ray powder diffraction patterns as compared to an X-ray powder diffraction pattern of pure Polymorph A.

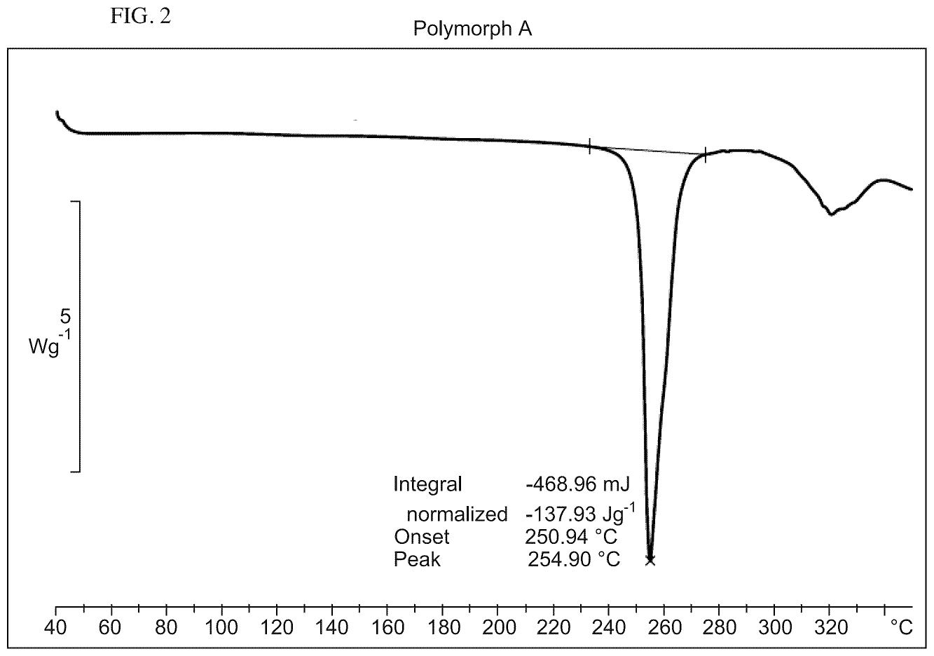

In certain embodiments, Polymorph A is identifiable on the basis of a characteristic peak observed in a differential scanning calorimetry thermogram. Differential scanning calorimetry, or DSC, is a thermoanalytical technique in which the difference in the amount of heat required to increase the temperature of a sample and reference is measured as a function of temperature. In some embodiments, Polymorph A exhibits a differential scanning calorimetry thermogram having a characteristic peak expressed in units of ° C. at a temperature of about 255+/−5° C. In some embodiments, Polymorph A exhibits a differential scanning calorimetry thermogram having a single endothermic peak observed at the temperature range of 250-255° C. In some embodiments, Polymorph A exhibits a differential scanning calorimetry thermogram substantially in accordance with FIG. 2.

In some embodiments, the crystalline form may contain impurities. Non-limiting examples of impurities include undesired polymorph forms, or residual organic and inorganic molecules such as solvents, water or salts. In some embodiments, the crystalline form is substantially free from impurities. In some embodiments, the crystalline form contains less than 10% by weight total impurities. In some embodiments, the crystalline form contains less than 5% by weight total impurities. In some embodiments, the crystalline form contains less than 1% by weight total impurities. In some embodiments, the crystalline form contains less than 0.1% by weight total impurities.

In some embodiments, the crystalline form has a purity of at least 99.8%. In some embodiments, the crystalline form has a purity of 99.8%. In some embodiments, the crystalline form has a purity of 99.9%. In some embodiments, the crystalline form has a purity of at least 95%, 96%, 97%, 98% or 99%. For example, in some embodiments, the crystalline form has a purity of at least 99.5%, 99.6%, 99.7%, 99.8%, or 99%.

In some embodiments, the crystalline form of Compound I hydrobromide contains less than 0.50%, less than 0.45%, less than 0.40%, less than 0.35%, less than 0.30%, less than 0.25%, less than 0.20%, less than 0.15%, less than 0.10%, or, less than 0.05% of N-dealkylation decomposition impurities. In some embodiments, the crystalline form of Compound I hydrobromide contains less than 0.2% of derivatives of Compound I. In some embodiments, the crystalline form of Compound I hydrobromide contains less than 0.2% of N-dealkylation decomposition impurities. In some embodiments, the crystalline form of Compound I hydrobromide contains less than 0.08%, 0.07%, 0.06%, 0.05%, 0.04%, 0.03% of N-dealkylation decomposition impurities.

In some embodiments, the crystalline form of Compound I hydrobromide is a crystalline solid substantially free of amorphous Compound I hydrobromide. As used herein, the term “substantially free of amorphous Compound I hydrobromide” means that the compound contains no significant amount of amorphous Compound I hydrobromide. In some embodiments, at least about 95% by weight of crystalline the crystalline form of Compound I hydrobromide is present.

In some embodiments of the disclosure, at least about 99% by weight of crystalline the crystalline form of Compound I hydrobromide is present.

In some embodiments, Polymorph A is a crystalline solid substantially free of amorphous Compound I hydrobromide. As used herein, the term “substantially free of amorphous Compound I hydrobromide” means that the compound contains no significant amount of amorphous Compound I hydrobromide. In some embodiments, at least about 95% by weight of crystalline Polymorph A is present. In some embodiments of the disclosure, at least about 99% by weight of crystalline Polymorph A is present.

In some embodiments, Polymorph A is substantially free of other polymorph forms of Compound I hydrobromide. In some embodiments, Polymorph A contains less than 0.5% of other polymorph forms of Compound I hydrobromide. In some embodiments, polymorph A contains less than 0.4%, less than 0.3%, less than 0.2%, or less than 0.1% of other polymorph forms of Compound I hydrobromide. In some embodiments, Polymorph A is free of other polymorph forms of Compound I hydrobromide.

In some embodiments, Polymorph A is substantially free of Polymorph B. As used herein, the term “substantially free of Polymorph B” means that the polymorph contains no significant amount of Polymorph B. In some embodiments, Polymorph A contains less than 0.5% of Polymorph B. In some embodiments, Polymorph A contains less than 0.4%, less than 0.3%, less than 0.2%, or less than 0.1% of Polymorph B. In some embodiments, Polymorph A is free of Polymorph B.

In some embodiments, the crystalline form of the disclosure, e.g. Polymorph A, can be found together with other substances or can be isolated. In some embodiments, the crystalline form of the disclosure, is substantially isolated. By “substantially isolated” is meant that a crystalline form is at least partially or substantially separated from the environment in which it was formed or detected. Partial separation can include, for example, a composition enriched in the salt of the disclosure. Substantial separation can include compositions containing at least about 50%, at least about 60%, at least about 70%, at least about 80%, at least about 90%, at least about 95%, at least about 97%, or at least about 99% by weight of the hydrobromide of Compound I. Methods for isolating compounds and their salts are routine in the art.

The hydrobromide of Compound I can occur as any reasonable tautomer, or a mixture of reasonable tautomers. As used herein, “tautomer” refers to one of two or more structural isomers that exist in equilibrium and are readily converted from one isomeric form to another. Examples include keto-enol tautomers, such as acetone/propen-2-ol, and the like. The hydrobromide of Compound I can have one or more tautomers and therefore include various isomers, i.e., pyridin-2(1H)-one and the corresponding pyridin-2-ol. All such isomeric forms of these compounds are expressly included in the present disclosure.

In some embodiments, the crystalline form of Compound I hydrobromide forms particles having a D90 particle size of from about 15 μm to about 50 μm. For example, in some embodiments, the crystalline form of Compound I hydrobromide forms particles having a D90 particle size of about 15 μm, about 20 μm, about 25 μm, about 30 μm, about 35 μm, about 40 μm, about 45 μm, or about 50 am.

In some embodiments, the crystalline form of Compound I hydrobromide forms particles having a D90 particle size of from about 25 μm to about 37 μm, from about 27 μm to about 35 μm, or 29 μm to about 33 am. For example, in some embodiments, the crystalline form of Compound I hydrobromide forms particles having a D90 particle size of about 25 μm, about 26 μm, about 27 μm, about 28 μm, about 29 μm, about 30 μm, about 31 μm, about 32 μm, about 33 μm, about 34 μm, about 35 μm, about 36 μm, or about 37 μm.

In some embodiments, the crystalline form of Compound I hydrobromide forms particles having a D10 particle size of from about 1 μm to about 15 μm, from about 3 μm to about 12 μm, or from about 5 μm to about 10 am. For example, in some embodiments, the crystalline form of Compound I hydrobromide forms particles having a D10 particle size of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm.

In some embodiments, the crystalline form of Compound I hydrobromide forms particles having a D50 particle size of from about 5 μm to about 25 μm, or from about 10 μm to about 20 μm. For example, in some embodiments, the crystalline form of Compound I hydrobromide forms particles having a D50 particle size of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm.

In some embodiments, provided herein is a crystalline form of Compound I hydrobromide wherein the crystalline form forms particles wherein at least about 50% of the particles have a particle size of from about 6 μm to about 40 μm. For example, in some embodiments, at least about 50%, at least about 60%, at least about 70%, at least about 80%, or at least about 90% of the particles have a particle size of from about 6 μm to about 40 μm.

In some embodiments, provided herein is a crystalline form of Compound I hydrobromide wherein the crystalline form forms particles wherein at least about 90% of the particles have a particle size of from about 6 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 6 μm to about 40 μm. In some embodiments, about 100% of the particles have a particle size of from about 6 μm to about 40 μm.

In some embodiments, provided herein is a crystalline form of Compound I hydrobromide wherein the crystalline form forms particles wherein at least about 50% of the particles have a particle size of from about 5 μm to about 50 μm. For example, in some embodiments, at least about 50%, at least about 60%, at least about 70%, at least about 80%, or at least about 90% of the particles have a particle size of from about 5 μm to about 50 μm.

In some embodiments, provided herein is a crystalline form of Compound I hydrobromide wherein the crystalline form forms particles wherein at least 90% of the particles have a particle size of from about 5 μm to about 50 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 5 μm to about 50 μm. In some embodiments, about 100% of the particles have a particle size of from about 5 μm to about 50 μm.

In some embodiments, provided herein is a crystalline form of Compound I hydrobromide wherein the crystalline form forms particles wherein at least about 50% of the particles have a particle size of from about 10 μm to about 40 μm. For example, in some embodiments, at least about 50%, at least about 60%, at least about 70%, at least about 80%, or at least about 90% of the particles have a particle size of from about 10 μm to about 40 μm.

In some embodiments, provided herein is a crystalline form of Compound I hydrobromide wherein the crystalline form forms particles wherein at least 90% of the particles have a particle size of from about 10 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 10 μm to about 40 μm. In some embodiments, about 100% of the particles have a particle size of from about 10 μm to about 40 μm.

In some embodiments, provided herein is a crystalline form of Compound I hydrobromide wherein the crystalline form forms particles wherein at least about 50% of the particles have a particle size of from about 15 μm to about 40 μm. For example, in some embodiments, at least about 50%, at least about 60%, at least about 70%, at least about 80%, or at least about 90% of the particles have a particle size of from about 15 μm to about 40 μm.

In some embodiments, provided herein is a crystalline form of Compound I hydrobromide wherein the crystalline form forms particles wherein at least 90% of the particles have a particle size of from about 15 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 15 μm to about 40 μm. In some embodiments, about 100% of the particles have a particle size of from about 15 μm to about 40 μm.

In some embodiments, provided herein is a crystalline form of Compound I hydrobromide wherein the crystalline form forms particles wherein at least about 50% of the particles have a particle size of from about 15 μm to about 35 μm. For example, in some embodiments, at least about 50%, at least about 60%, at least about 70%, at least about 80%, or at least about 90% of the particles have a particle size of from about 15 μm to about 35 μm.

In some embodiments, provided herein is a crystalline form of Compound I hydrobromide wherein the crystalline form forms particles wherein at least 90% of the particles have a particle size of from about 15 μm to about 35 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 15 μm to about 35 μm. In some embodiments, about 100% of the particles have a particle size of from about 15 μm to about 35 μm.

In some embodiments, provided herein is a crystalline form of Compound I hydrobromide wherein the crystalline form forms particles wherein at least about 50% of the particles have a particle size of from about 20 μm to about 35 μm. For example, in some embodiments, at least about 50%, at least about 60%, at least about 70%, at least about 80%, or at least about 90% of the particles have a particle size of from about 20 μm to about 35 μm.

In some embodiments, provided herein is a crystalline form of Compound I hydrobromide wherein the crystalline form forms particles wherein at least 90% of the particles have a particle size of from about 20 μm to about 35 μm. For example, in some embodiments, at least about 90%, at least about 91%, at least about 92%, at least about 93%, at least about 94%, at least about 95%, at least about 96%, at least about 97%, at least about 98%, or at least about 99% of the particles have a particle size of from about 20 μm to about 35 μm. In some embodiments, about 100% of the particles have a particle size of from about 20 μm to about 35 μm.

In some embodiments, provided herein is a crystalline form of Compound I hydrobromide, wherein the crystalline form forms particles wherein at least about 60%, at least about 70%, at least about 80%, at least about 90%, or at least about 95% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm. In some embodiments, provided herein is a crystalline form of Compound I hydrobromide, wherein the crystalline form forms particles wherein about 100% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.

In some embodiments, the crystalline form of the disclosure forms particles wherein the particles have a particle size distribution with a relative span of from about 1 to about 5, or from about 2 to about 4. In some embodiments, the crystalline form of the disclosure forms particles wherein the particles have a particle size distribution with a relative span of from about 1 to about 2. For example, in some embodiments, the crystalline form of the disclosure forms particles wherein the particles a particle size distribution with a relative span of about 1.0, about 1.1, about 1.2, about 1.3, about 1.4, about 1.5, about 1.6, about 1.7, about 1.8, about 1.9, or about 2.0. In some embodiments, the crystalline form of the disclosure forms particles wherein the particles a particle size distribution with a relative span of about 2.5, about 2.7, or about 3.0.

In some embodiments, provided herein is a plurality of microparticles of Compound I hydrobromide wherein at least about 50% of the microparticles have a particle size of from about 6 μm to about 40 μm. For example, in some embodiments, at least about 50%, at least about 60%, at least about 70%, at least about 80%, or at least about 90% of the microparticles have a particle size of from about 6 μm to about 40 μm.

In some embodiments, provided herein is a plurality of microparticles of Compound I hydrobromide wherein at least about 90% of the microparticles have a particle size of from about 6 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 6 μm to about 40 μm. In some embodiments, about 100% of the microparticles have a particle size of from about 6 μm to about 40 μm.

In some embodiments, provided herein is a plurality of microparticles of Compound I hydrobromide wherein at least about 50% of the microparticles have a particle size of from about 5 μm to about 50 μm. For example, in some embodiments, at least about 50%, at least about 60%, at least about 70%, at least about 80%, or at least about 90% of the microparticles have a particle size of from about 5 μm to about 50 μm.

In some embodiments, provided herein is a plurality of microparticles of Compound I hydrobromide wherein at least 90% of the microparticles have a particle size of from about 5 μm to about 50 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 5 μm to about 50 μm. In some embodiments, about 100% of the microparticles have a particle size of from about 5 μm to about 50 μm.

In some embodiments, provided herein is a plurality of microparticles of Compound I hydrobromide wherein at least about 50% of the microparticles have a particle size of from about 10 μm to about 40 μm. For example, in some embodiments, at least about 50%, at least about 60%, at least about 70%, at least about 80%, or at least about 90% of the microparticles have a particle size of from about 10 μm to about 40 μm.

In some embodiments, provided herein is a plurality of microparticles of Compound I hydrobromide wherein at least 90% of the microparticles have a particle size of from about 10 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 10 μm to about 40 μm. In some embodiments, about 100% of the microparticles have a particle size of from about 10 μm to about 40 μm.

In some embodiments, provided herein is a plurality of microparticles of Compound I hydrobromide wherein at least about 50% of the microparticles have a particle size of from about 15 μm to about 40 μm. For example, in some embodiments, at least about 50%, at least about 60%, at least about 70%, at least about 80%, or at least about 90% of the microparticles have a particle size of from about 15 μm to about 40 μm.

In some embodiments, provided herein is a plurality of microparticles of Compound I hydrobromide wherein at least 90% of the microparticles have a particle size of from about 15 μm to about 40 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 15 μm to about 40 μm. In some embodiments, about 100% of the microparticles have a particle size of from about 15 μm to about 40 μm.

In some embodiments, provided herein is a plurality of microparticles of Compound I hydrobromide wherein at least about 50% of the microparticles have a particle size of from about 15 μm to about 35 μm. For example, in some embodiments, at least about 50%, at least about 60%, at least about 70%, at least about 80%, or at least about 90% of the microparticles have a particle size of from about 15 μm to about 35 μm.

In some embodiments, provided herein is a plurality of microparticles of Compound I hydrobromide wherein at least 90% of the microparticles have a particle size of from about 15 μm to about 35 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 15 μm to about 35 μm. In some embodiments, about 100% of the microparticles have a particle size of from about 15 μm to about 35 μm.

In some embodiments, provided herein is a plurality of microparticles of Compound I hydrobromide wherein at least about 50% of the microparticles have a particle size of from about 20 μm to about 35 μm. For example, in some embodiments, at least about 50%, at least about 60%, at least about 70%, at least about 80%, or at least about 90% of the microparticles have a particle size of from about 20 μm to about 35 μm.

In some embodiments, provided herein is a plurality of microparticles of Compound I hydrobromide wherein at least 90% of the microparticles have a particle size of from about 20 μm to about 35 μm. For example, in some embodiments, at least about 90%, at least about 91%, at least about 92%, at least about 93%, at least about 94%, at least about 95%, at least about 96%, at least about 97%, at least about 98%, or at least about 99% of the microparticles have a particle size of from about 20 μm to about 35 μm. In some embodiments, about 100% of the microparticles have a particle size of from about 20 μm to about 35 μm.

In some embodiments, provided herein is a plurality of microparticles of Compound I hydrobromide, wherein at least about 60%, at least about 70%, at least about 80%, at least about 90%, or at least about 95% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm. In some embodiments, provided herein is a plurality of microparticles of Compound I hydrobromide, wherein about 100% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.

In some embodiments, the microparticles have a particle size distribution with a relative span of from about 1 to about 5, or from about 2 to about 4. In some embodiments, the microparticles have a particle size distribution with a relative span of from about 1 to about 2.

For example, in some embodiments, the microparticles have a particle size distribution with a relative span of about 1.0, about 1.1, about 1.2, about 1.3, about 1.4, about 1.5, about 1.6, about 1.7, about 1.8, about 1.9, or about 2.0. In some embodiments, the microparticles have a particle size distribution with a relative span of about 2.5, about 2.7, or about 3.0.

In some embodiments, the crystalline form of Compound I hydrobromide has a residual ethanol solvent content of about 5000 ppm or less. For example, in some embodiments, the crystalline form of Compound I hydrobromide has a residual ethanol solvent content of about 4500 ppm or less, about 4000 ppm or less, about 3500 ppm or less, about 3000 ppm or less, about 2500 ppm or less, about 2000 ppm or less, about 1500 ppm or less, about 1000 ppm or less, or about 500 ppm or less. In some embodiments, the crystalline form of Compound I hydrobromide has a residual ethanol solvent content of about 3720 ppm.

In some embodiments, the crystalline form of Compound I hydrobromide has a residual ethanol solvent content of about 350 ppm or less. For example, in some embodiments, the crystalline form of Compound I hydrobromide has a residual ethanol solvent content of about 300 ppm or less, about 250 ppm or less, about 200 ppm or less, about 150 ppm or less, about 100 ppm or less, or about 50 ppm or less. In some embodiments, the crystalline form of Compound I hydrobromide has a residual ethanol solvent content of about 320 ppm or less. In some embodiments, the crystalline form of Compound I hydrobromide has a residual ethanol solvent content of about 320 ppm. In some embodiments, the crystalline form of Compound I hydrobromide is substantially free of residual ethanol solvent.

In some embodiments, the crystalline form of Compound I hydrobromide has a residual ethyl acetate solvent content of about 5000 ppm or less. For example, in some embodiments, the crystalline form of Compound I hydrobromide has a residual ethyl acetate solvent content of about 4500 ppm or less, about 4000 ppm or less, about 3500 ppm or less, about 3000 ppm or less, about 2500 ppm or less, about 2000 ppm or less, about 1500 ppm or less, about 1000 ppm or less, or about 500 ppm or less. In some embodiments, the crystalline form of Compound I hydrobromide has a residual ethyl acetate solvent content of 2764 ppm or less. In some embodiments, the crystalline form of Compound I hydrobromide has a residual ethyl acetate solvent content of about 2764 ppm.

In some embodiments, the crystalline form of Compound I hydrobromide has a residual ethyl acetate solvent content of about 100 ppm or less. For example, in some embodiments, the crystalline form of Compound I hydrobromide has a residual ethyl acetate solvent content of about 80 ppm or less, about 75 ppm or less, about 70 ppm or less, about 65 ppm or less, about 60 ppm or less, about 55 ppm or less, about 50 ppm or less, about 45 ppm or less, about 40 ppm or less, about 35 ppm or less, about 30 ppm or less, about 25 ppm or less, about 20 ppm or less, about 15 ppm or less, or about 10 ppm or less. In some embodiments, the crystalline form of Compound I hydrobromide has a residual ethyl acetate solvent content of about 75 ppm or less.

In some embodiments, the crystalline form of Compound I hydrobromide has a residual ethyl acetate solvent content of about 75 ppm. In some embodiments, the crystalline form of Compound I hydrobromide is substantially free of residual ethyl acetate solvent.

In some embodiments, the crystalline form of Compound I hydrobromide has a residual toluene solvent content of about 890 ppm or less. For example, in some embodiments, the crystalline form of Compound I hydrobromide has a residual toluene solvent content of about 800 ppm or less, about 700 ppm or less, about 600 ppm or less, about 500 ppm or less, about 400 ppm or less, about 300 ppm or less, about 200 ppm or less, about 100 ppm or less, or about 50 ppm or less. In some embodiments, the crystalline form of Compound I hydrobromide has a residual toluene solvent content of about 84 ppm.

In some embodiments, the crystalline form of Compound I hydrobromide has a residual toluene solvent content of about 25 ppm or less. For example, in some embodiments, the crystalline form of Compound I hydrobromide has a residual toluene solvent content of about 20 ppm or less, about 15 ppm or less, about 10 ppm or less, or about 5 ppm or less. In some embodiments, the crystalline form of Compound I hydrobromide has a residual toluene solvent content of about 20 ppm. In some embodiments, the crystalline form of Compound I hydrobromide is substantially free of residual toluene solvent.

As used herein the “D90 particle size” as means the particle size at the 90% percentile. In other words, “D90” describes a diameter where 90% of the particles have a smaller particle diameter than the stated value. The term “90% cumulative particle diameter in particle size distribution” is synonymous with “D90.”

As used herein the “span” and the “relative span” of the particle size distribution are statistical parameters describing the width of the particle size distribution. Mathematically, the span is the difference between the D90 and the D10 values (D90−D10). The relative span is described as follows: Relative span=(D90−D10)/D50.

### Pharmaceutical Compositions

In some aspects, provided herein is a pharmaceutical composition comprising crystalline particles comprising a crystalline form of Compound I hydrobromide of the disclosure, and a pharmaceutically acceptable carrier or diluent.

In some aspects, provided herein is a pharmaceutical composition comprising crystalline particles comprising crystalline particles of a polymorph of Compound I hydrobromide of the disclosure, and a pharmaceutically acceptable carrier or diluent.

In some embodiments, provided herein is a pharmaceutical composition comprising a plurality of microparticles of Compound I hydrobromide and a pharmaceutically acceptable carrier or diluent.

In some aspects, provided herein is a pharmaceutical composition comprising crystalline particles comprising a polymorph of Compound I hydrobromide prepared by a method of the disclosure, and a pharmaceutically acceptable carrier or diluent.

In some aspects, provided herein is a pharmaceutical composition comprising crystalline particles comprising crystalline particles of a polymorph of Compound I hydrobromide prepared by a method of the disclosure, and a pharmaceutically acceptable carrier or diluent.

In some embodiments, the pharmaceutical composition is in a solid unit dosage form. In some embodiments, the pharmaceutical composition is an oral unit dosage form. In some embodiments, the pharmaceutical composition is in the form of a tablet.

The present disclosure also relates to a solid pharmaceutical composition comprising a polymorph of Compound I hydrobromide (e.g. in the form of crystalline particles) and one or more pharmaceutically acceptable excipients selected from sodium starch glycolate, carmellose, carmellose calcium, croscarmellose sodium, or low-substituted hydroxypropylcellulose, and a combination thereof. In some embodiments, the excipients are selected from sodium starch glycolate, carmellose, carmellose calcium, or croscarmellose sodium, and a combination thereof.

In some embodiments, the excipients are selected from sodium starch glycolate, or carmellose, and a combination thereof. In some embodiments, the solid pharmaceutical composition further includes lactose, hydroxypropyl cellulose, or magnesium stearate or a combination thereof.

In some embodiments, the solid pharmaceutical composition further includes lactose monohydrate, low-substituted hydroxypropyl cellulose, hydroxypropyl cellulose, sodium starch glycolate, and magnesium stearate.

The composition of the disclosure can include one or more of the following features when applicable:

In some embodiments, the concentration of a crystalline form of Compound I hydrobromide in the composition is from about 30 wt. % to about 70 wt. %, from about 40 wt. % to about 70 wt. %, or from about 50 wt. % to about 60 wt. %. In some embodiments, the concentration of a crystalline form of Compound I hydrobromide in the composition is about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, or about 60 wt. %. In some embodiments, the concentration of a crystalline form of Compound I hydrobromide in the composition is 57.1 wt. %.

In some embodiments, the one or more pharmaceutically acceptable excipients include a diluent(s), a disintegrant(s), and a binder(s).

In some embodiments, the composition comprises from about 10 wt. % to about 20 wt. % diluent. In some embodiments, the composition comprises about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. % diluent.

In some embodiments, the diluent is lactose monohydrate.

In some embodiments, the composition comprises from about 10 wt. % to about 20 wt. % lactose monohydrate. In some embodiments, the composition comprises about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. % lactose monohydrate.

In some embodiments, the composition comprises from about 15 wt. % to about 25 wt. % disintegrant. In some embodiments, the composition comprises about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, or about 25 wt. % disintegrant.

In some embodiments, the disintegrant comprises low-substituted hydroxypropyl cellulose, sodium starch glycolate, or a combination thereof.

In some embodiments, the composition comprises from about 10 wt. % to about 20 wt. % low-substituted hydroxypropyl cellulose. In some embodiments, the composition comprises about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. % low-substituted hydroxypropyl cellulose.

In some embodiments, the composition comprises from about 1 wt. % to about 10 wt. % sodium starch glycolate. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % sodium starch glycolate.

In some embodiments, the composition comprises from about 1 wt. % to about 10 wt. % binder. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % binder.

In some embodiments, the binder is hydroxypropyl cellulose.

In some embodiments, the composition comprises from about 1 wt. % to about 10 wt. % hydroxypropyl cellulose. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % hydroxypropyl cellulose.

In some embodiments, the one or more pharmaceutically acceptable excipients further comprise a lubricant.

In some embodiments, the composition comprises from about 0.5 wt. % to about 5 wt. % lubricant. In some embodiments, the composition comprises about 0.5 wt. %, about 0.7 wt. %, about 0.9 wt. %, about 1 wt. %, about 2 wt. %, about 3 wt. %, about 4 wt. %, or about 5 wt. % lubricant

In some embodiments, the lubricant is magnesium stearate.

In some embodiments, the composition comprises from about 0.5 wt. % to about 5 wt. % magnesium stearate. In some embodiments, the composition comprises about 0.5 wt. %, about 0.7 wt. %, about 0.9 wt. %, about 1 wt. %, about 2 wt. %, about 3 wt. %, about 4 wt. %, or about 5 wt. % magnesium stearate.

In some embodiments, the one or more pharmaceutically acceptable excipients further comprise a coating composition.

In some embodiments, the pharmaceutical composition comprises a crystalline form of Compound I hydrobromide in an amount of about 50-60 wt. %; lactose monohydrate in an amount of about 10-20 wt. %; low-substituted hydroxypropyl cellulose in an amount of about 11-19 wt. %; sodium starch glycolate in an amount of about 3-7 wt. %; hydroxypropyl cellulose in an amount of about 1-10 wt. %; and magnesium stearate in an amount of about 0.5-5 wt. %.

In some embodiments, the pharmaceutical composition comprises a crystalline form of Compound I hydrobromide in an amount of about 57 wt. %; lactose monohydrate in an amount of about 17 wt. %; low-substituted hydroxypropyl cellulose in an amount of about 15 wt. %; sodium starch glycolate in an amount of about 5 wt. %; hydroxypropyl cellulose in an amount of about 4 wt. %; and magnesium stearate in an amount of about 2 wt. %.

In some embodiments, the pharmaceutical composition comprises a crystalline form of Compound I hydrobromide in an amount of about 50-60 wt. %; lactose monohydrate in an amount of about 10-20 wt. %; low-substituted hydroxypropyl cellulose in an amount of about 11-19 wt. %; sodium starch glycolate in an amount of about 3-7 wt. %; hydroxypropyl cellulose in an amount of about 1-10 wt. %; and magnesium stearate in an amount of about 0.5-5 wt. % and a coating composition in an amount of about 1-10 wt. %.

In some embodiments, the composition comprises from about 1 wt. % to about 10 wt. % coating composition. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % coating composition.

In some embodiments, the coating composition is a water-soluble, immediate-release coating composition.

In some embodiments, the coating composition is a hydroxypropyl methylcellulose-based film coating.

In some embodiments, the coating composition comprises hypromellose.

In some embodiments, the coating composition further comprises talc. In some embodiments, the coating composition further comprises macrogol.

In some embodiments, the coating composition further comprises a colorant. In some embodiments, the composition comprises titanium dioxide, iron (III) oxide, or both.

In some embodiments, the coating composition further comprises a colorant. In some embodiments, the composition comprises titanium dioxide, iron(III) oxide-hydroxide, or both.

In some embodiments, the coating composition comprises one or more of polyvinyl alcohol, hypromellose, talc, and macrogol. In some embodiments, the coating composition further comprises titanium dioxide and/or iron (III) oxide. In some embodiments, the coating composition is an Opadry® film coating. In some embodiments, the coating composition is Opadry® 03F45063 RED. In some embodiments, the coating composition is Opadry® 03F220119 YELLOW.

In some embodiments, the composition comprises about 1 wt. % to about 10 wt. % Opadry® 03F45063 RED. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % Opadry® 03F45063 RED.

In some embodiments, the composition comprises about 1 wt. % to about 10 wt. % Opadry® 03F220119 YELLOW. In some embodiments, the composition comprises about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % Opadry® 03F220119 YELLOW.

In some embodiments, the composition comprises a crystalline form of Compound I hydrobromide in an amount of about 50-60 wt. %, about 10-20 wt. % diluent, about 15-25 wt. % disintegrant, about 1-10 wt. % binder, about 0.5-5 wt. % lubricant, and about 1-10 wt. % coating composition. In some embodiments, the composition comprises a crystalline form of Compound I hydrobromide, in an amount of about 50-60 wt. %, about 12-18 wt. % diluent, about 18-23 wt. % disintegrant, about 2-6 wt. % binder, about 1-3 wt. % lubricant, and about 2-6 wt. % coating composition.

In some embodiments, the composition consists of the therapeutic agent, lactose monohydrate, low-substituted hydroxypropyl cellulose, sodium starch glycolate, hydroxypropyl cellulose, and magnesium stearate. In some embodiments, the composition consists of a crystalline form of Compound I hydrobromide in an amount of about 50-60 wt. %, about 10-20 wt. % lactose monohydrate, about 11-19 wt. % low-substituted hydroxypropyl cellulose, about 3-7 wt. % sodium starch glycolate, about 1-10 wt. % hydroxypropyl cellulose, and about 0.5-5 wt. % magnesium stearate. In some embodiments, the composition consists of a crystalline form of Compound I hydrobromide in an amount of about 55 wt. %, about 17 wt. % lactose monohydrate, about 15 wt. % low-substituted hydroxypropyl cellulose, about 5 wt. % sodium starch glycolate, about 4 wt. % hydroxypropyl cellulose, and about 2 wt. % magnesium stearate.

In some embodiments, the composition consists of the therapeutic agent, lactose monohydrate, low-substituted hydroxypropyl cellulose, sodium starch glycolate, hydroxypropyl cellulose, magnesium stearate, and a coating composition. In some embodiments, the composition consists of a crystalline form of Compound I hydrobromide in an amount of about 50-60 wt. %, about 10-20 wt. % lactose monohydrate, about 11-19 wt. % low-substituted hydroxypropyl cellulose, about 3-7 wt. % sodium starch glycolate, about 1-10 wt. % hydroxypropyl cellulose, about 0.5-5 wt. % magnesium stearate, and about 1-10 wt. % a coating composition. In some embodiments, the composition consists of a crystalline form of Compound I hydrobromide in an amount of about 50-60 wt. %, about 16 wt. % lactose monohydrate, about 14-15 wt. % low-substituted hydroxypropyl cellulose, about 5 wt. % sodium starch glycolate, about 4 wt. % hydroxypropyl cellulose, about 2 wt. % magnesium stearate, and about 4 wt. % a coating composition.

In some embodiments, the composition comprises one or more additional therapeutic agents.

In some embodiments, the composition is an oral dosage composition comprising an amount of a crystalline form of Compound I hydrobromide in an amount equivalent to from about 10 mg to about 1000 mg, from about 10 mg to about 800 mg, from about 10 mg to about 500 mg, or from about 10 mg to about 400 mg of Compound I. In some embodiments, the oral dosage composition comprises a crystalline form of Compound I hydrobromide in an amount of about 28.5, about 57 mg, about 114 mg, about 228, or about 456 mg of Compound I hydrobromide. In some embodiments, the oral dosage composition is in the form of a tablet. In some embodiments, the tablet comprises a crystalline form of Compound I hydrobromide in an amount of from about 25 mg to about 500 mg. In some embodiments, the tablet comprises a crystalline form of Compound I hydrobromide in an amount equivalent to about 50 mg, about 75 mg, about 100 mg, about 125 mg, about 150 mg, about 175 mg, about 200 mg or about 400 mg of Compound I. In some embodiments, the tablet comprises a crystalline form of Compound I hydrobromide in an amount of about 28.5 mg, about 57 mg, about 114 mg, about 228, or about 456 mg.

In some embodiments, the composition is a solid composition. In some embodiments, the composition is substantially free of water. In this context, “substantially” free of water means that the water content of the composition at the time of packaging is less than 7%, less than 5%, less than 1%, or less than 0.5% of the total weight of the composition. In some embodiments the amount of water is between 0.1 to 5% (e.g., 0.1-1% or 0.1-0.5%) of the total weight of the composition. In some embodiments, the amount of water in the composition of the disclosure manufactured through a spray-coating process is less than 0.5%. In some embodiments, the present disclosure relates to an oral composition (e.g., in the form of a tablet) which is a stable composition. For example, a stable composition of the disclosure retains an amount of the active compound (e.g., Compound I or a salt thereof) in the composition over a period of time (e.g., 3 months, 12 months, 18 months and 24 months), that is at least 90%, preferably at least 95%, and most preferably at least 99% the amount of the active compound initially present in the composition. The storage condition can be 2-8 degrees Celsius (2-8° C.), or 25 degrees Celsius (25° C.) and 60% relative humidity, or 25° C. and 75% relative humidity, or 40° C. and 75% relative humidity.

In some embodiments, the present disclosure relates to the pharmaceutical composition comprising a crystalline form of Compound I hydrobromide and one or more pharmaceutically acceptable excipients selected from sodium starch glycolate, carmellose, carmellose calcium, croscarmellose sodium, or low-substituted hydroxypropylcellulose, and a combination thereof. In some embodiments, the excipients are selected from sodium starch glycolate, carmellose, carmellose calcium, or croscarmellose sodium, and a combination thereof. In some embodiments, the excipients are selected from sodium starch glycolate, or carmellose, and a combination thereof. In some embodiments, the pharmaceutical composition further includes lactose, hydroxypropyl cellulose, or magnesium stearate or a combination thereof.

In some embodiments, the pharmaceutical composition comprises a crystalline form of Compound I hydrobromide in an amount of about 50-60 wt. % and about 5-35 wt. % excipients selected from sodium starch glycolate, carmellose, carmellose calcium, croscarmellose sodium, or low-substituted hydroxypropylcellulose, and a combination thereof. In some embodiments, the pharmaceutical composition comprises a crystalline form of Compound I hydrobromide in an amount of about 50-60 wt. %, about 10-30 wt. % excipients selected from sodium starch glycolate, carmellose, carmellose calcium, croscarmellose sodium, or low-substituted hydroxypropylcellulose, and a combination thereof, about 10-20 wt. % diluent, about 2-6 wt. % binder, and about 1-3 wt. % lubricant.

In some embodiments, the pharmaceutical composition comprises a crystalline form of Compound I hydrobromide in an amount of about 50-60 wt. %, about 20 wt. % excipients selected from sodium starch glycolate, carmellose, carmellose calcium, croscarmellose sodium, or low-substituted hydroxypropylcellulose, and a combination thereof, about 10-20 wt. % diluent, about 2-6 wt. % binder, and about 1-3 wt. % lubricant. In some embodiments, the formulation comprises a crystalline form of Compound I hydrobromide in an amount of about 50-60 wt. %, about 20 wt. % excipients selected from sodium starch glycolate, carmellose, and a combination thereof, about 10-20 wt. % lactose monohydrate, about 2-6 wt. % hydroxypropylcellulose, and about 1-3 wt. % magnesium stearate. The term “pharmaceutical composition” includes preparations suitable for administration to a subject. In some embodiments, the subject is a mammal, e.g., a human. When the compounds of the present disclosure are administered as pharmaceuticals to a subject (e.g., a mammal, e.g., a human), they can be given per se or as a pharmaceutical composition containing, for example, 0.1% to 99.9% (more preferably, 0.5 to 90%) of active ingredient in combination with a pharmaceutically acceptable carrier.

The compounds described herein (i.e., the hydrobromide of Compound I) can be combined with a pharmaceutically acceptable carrier according to conventional pharmaceutical compounding techniques. As used herein, “pharmaceutically acceptable carrier” may include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Furthermore, the carrier may take a wide variety of forms depending on the form of the preparation desired for administration, e.g. oral, nasal, rectal, vaginal, parenteral (including intravenous injections or infusions). In preparing compositions for oral dosage form any of the usual pharmaceutical media may be employed. Usual pharmaceutical media include, for example, water, glycols, oils, alcohols, flavoring agents, preservatives, coloring agents, and the like in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, tocopherols, and the like; and metal chelating agents, such as citric acid, ethylenediamine tetraacetic acid (EDTA), sorbitol, tartaric acid, phosphoric acid, and the like.

Pharmaceutical compositions comprising the compounds may be formulated to have any concentration desired. In some embodiments, the composition is formulated such that it comprises at least a therapeutically effective amount. In some embodiments, the composition is formulated such that it comprises an amount that would not cause one or more unwanted side effects.

Because crystalline forms of the hydrobromide of Compound I are more easily maintained during its preparation, solid dosage forms are a preferred form for the pharmaceutical composition of the disclosure. Solid dosage forms for oral administration, such as capsules, tablets, pills, powders, and granules, are particularly preferred. If desired, tablets may be coated by techniques known to those in the art.

Pharmaceutical compositions include those suitable for oral, sublingual, nasal rectal, vaginal, topical, buccal and parenteral (including subcutaneous, intramuscular, and intravenous) administration, although the most suitable route will depend on the nature and severity of the condition being treated. The compositions may be conveniently presented in unit dosage form, and prepared by any of the methods well known in the art of pharmacy. In some embodiments, the pharmaceutical composition is formulated for oral administration in the form of a pill, capsule, lozenge or tablet. In some embodiments, the pharmaceutical composition is in the form of a suspension.

The compounds provided herein are suitable as an active agent in pharmaceutical compositions that are efficacious particularly for treating EZH2-associated disorders, especially cancer. The pharmaceutical composition in various embodiments has a pharmaceutically effective amount of a crystalline form of the hydrobromide of Compound I or Polymorph A, along with other pharmaceutically acceptable excipients, carriers, fillers, diluents and the like.

A therapeutically or pharmaceutically “effective amount” is an amount of a compound (a crystalline form of the hydrobromide of Compound I or Polymorph A), that when administered to a patient, ameliorates a symptom of a disease or condition, e.g., prevent the various morphological and somatic symptoms of cancer. In an example, an effective amount of a crystalline form of the hydrobromide of Compound I or Polymorph A is the amount sufficient to treat cancer in a subject. The amount can vary depending on such factors as the size and weight of the subject, the type of illness, or the particular compound of the disclosure. The amount of a crystalline form of the hydrobromide of Compound I or Polymorph A that constitutes an “effective amount” will vary depending on the compound, the disease state and its severity, the age of the patient to be treated, and the like. The effective amount can be determined routinely by one of ordinary skill in the art having regard to their knowledge and to this disclosure.

The regimen of administration can affect what constitutes a pharmaceutically effective amount. A crystalline form of the hydrobromide of Compound I or Polymorph A, and compositions comprising either of these compounds, can be administered to the subject either prior to or after the onset of a disease. Further, several divided dosages, as well as staggered dosages can be administered daily or sequentially, or the dose can be continuously infused, or can be a bolus injection. Further, the dosages can be proportionally increased or decreased as indicated by the exigencies of the therapeutic or prophylactic situation.

### Methods of Treatment

Compounds of the present disclosure (i.e., a hydrobromide of Compound I) inhibit the histone methyltransferase activity of EZH2 or a mutant thereof and, accordingly, in some aspects of the disclosure, certain compounds disclosed herein are candidates for treating, or preventing certain conditions and diseases. The present disclosure provides methods for treating conditions and diseases the course of which can be influenced by modulating the methylation status of histones or other proteins, wherein said methylation status is mediated at least in part by the activity of EZH2. Modulation of the methylation status of histones can in turn influence the level of expression of target genes activated by methylation, and/or target genes suppressed by methylation. In some embodiments, the method includes administering to a subject in need of such treatment, a therapeutically effective amount of a compound of the present disclosure.

The disorder in which EZH2-mediated protein methylation plays a part can be cancer or a precancerous condition. The present disclosure further provides the use of a compound of the present disclosure (i.e., a crystalline form of the hydrobromide of Compound I) in the treatment of cancer or precancer the course of which can be influenced by modulating EZH2-mediated protein methylation, or, for the preparation of a medicament useful for the treatment of such cancer or pre-cancer. Exemplary cancers that may be treated include lymphomas, including non-Hodgkin lymphoma, follicular lymphoma (FL) and diffuse large B-cell lymphoma (DLBCL); melanoma; and leukemia, including CML. Exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullary carcinoma, and not otherwise specified (NOS) sarcoma. Alternatively, cancers to be treated by the compounds of the present disclosure are non NHL cancers.

Exemplary precancerous condition includes myelodysplastic syndrome (MDS; formerly known as preleukemia).

In some embodiments, provided herein is a method of treating a lymphoma comprising administering to the subject in need thereof an effective amount of a crystalline form of the hydrobromide of Compound I.

In some embodiments, the cancer is epithelioid sarcoma.

In some embodiments, the cancer is follicular lymphoma. In some embodiments, the cancer is a relapsed or refractory follicular lymphoma.

In some embodiments, the cancer is prostate cancer.

In some embodiments, the cancer is breast cancer.

In some embodiments, the breast cancer is estrogen receptor (ER) negative. In some embodiments, the breast cancer is progesterone receptor (PR) negative. In some embodiments, the breast cancer is HER2 negative. In some embodiments, the breast cancer is progesterone receptor (PR) negative. In some embodiments, the breast cancer is HER2 negative, estrogen receptor (ER) negative. In some embodiments, the breast cancer is HER2 negative, estrogen receptor (ER) negative and progesterone receptor (PR) negative. In some embodiments, the cancer is triple negative breast cancer.

In some embodiments, the cancer is ovarian cancer.

The present disclosure also provides methods of protecting against a disorder in which EZH2-mediated protein methylation plays a part in a subject in need thereof by administering a therapeutically effective amount of compound of the present disclosure (i.e., a crystalline form of the hydrobromide of Compound I, as well as Polymorph A) to a subject in need of such treatment. The disorder can be cancer, e.g., cancer in which EZH2-mediated protein methylation plays a role. The present disclosure also provides the use of compound of the present disclosure (i.e., a crystalline form of the hydrobromide of Compound I, as well as Polymorph A) for the preparation of a medicament useful for the prevention of a cell proliferative disorder associated, at least in part, with EZH2-mediated protein methylation.

The compounds of this disclosure can be used to modulate protein (e.g., histone) methylation, e.g., to modulate histone methyltransferase or histone demethylase enzyme activity. At least some of the compounds of the disclosure can be used in vivo or in vitro for modulating protein methylation. Histone methylation has been reported to be involved in aberrant expression of certain genes in cancers, and in silencing of neuronal genes in non-neuronal cells. At least some compounds described herein are suitable candidates for treating these diseases, i.e., to decreases methylation or restores methylation to roughly its level in counterpart normal cells.

Compounds that are methylation modulators may be used for modulating cell proliferation. For example, in some cases excessive proliferation may be reduced with agents that decrease methylation, whereas insufficient proliferation may be stimulated with agents that increase methylation. Accordingly, diseases that may be treated by the compounds of the disclosure can include hyperproliferative diseases, such as benign cell growth and malignant cell growth.

A subject in need thereof may have refractory or resistant cancer. “Refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

“Relapsed and/or refractory cancer” refers to a cancer unresponsive to a drug or a therapy. For example and without limitation, relapsed and/or refractory cancer includes cancer in patients whose first progression occurs in the absence of any treatment following successful treatment with a drug or a therapy; cancer in patients who progress within 60 days of the treatment; and cancer in patients who progress while receiving treatment, e.g., a standard-of care treatment.

The disclosure also provides methods for combination therapy in which a hydrobromide of Compound I (e.g., Polymorph A) and one or more other therapeutic agents are administered to a subject in need for treatment of a disease or cancer. The combination therapy can also be administered to cancer cells to inhibit proliferation or induce cell death. In some aspects a crystalline form of the hydrobromide of Compound I (e.g., Polymorph A) is administered subsequent to administration of the one or more other therapeutic agents. In some aspects, a crystalline form of the hydrobromide of Compound I is administered prior to administration of the one or more other therapeutic agents. In some aspects, a crystalline form of the hydrobromide of Compound I (e.g., Polymorph A) is administered subsequent to administration of one or more therapeutic agents, such that the other therapeutic agents are administered either in a single composition or in two or more compositions, e.g. administered simultaneously, sequentially, or in alternation. In some aspects, a crystalline form of the hydrobromide of Compound I (e.g., Polymorph A) is administered prior to administration of one or more therapeutic agents, such that the other therapeutic agents are administered either in a single composition or in two or more compositions, e.g. administered simultaneously, sequentially, or in alternation.

In some embodiments, “combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation.

In some aspects of the disclosure, the combination therapies featured in the disclosure can result in a synergistic effect in the treatment of a disease or cancer. A “synergistic effect” is defined as where the efficacy of a combination of therapeutic agents is greater than the sum of the effects of any of the agents given alone. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

In some aspects of the disclosure “combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some aspects, a composition of the disclosure, or a pharmaceutically acceptable salt, solvate, analog or derivative thereof, may be administered in combination with radiation therapy. Radiation therapy can also be administered in combination with a composition of the disclosure and another chemotherapeutic agent described herein as part of a multiple agent therapy.

Combination therapy can be achieved by administering two or more agents, e.g., a crystalline form of the hydrobromide of Compound I (e.g., Polymorph A) and one or more other therapeutic agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

As used herein, a “subject in need thereof” is a subject having a disorder in which EZH2-mediated protein methylation plays a part, or a subject having an increased risk of developing such disorder relative to the population at large. A subject in need thereof can have a precancerous condition. Preferably, a subject in need thereof has cancer. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human.

As used herein, the term “cell proliferative disorder” refers to conditions in which unregulated or abnormal growth, or both, of cells can lead to the development of an unwanted condition or disease, which may or may not be cancerous. Exemplary cell proliferative disorders that may be treated with the compounds of the disclosure encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. In some aspects, the methods provided herein are used to treat or alleviate a symptom of cancer or to identify suitable candidates for such purposes. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders that may be treated using one or more compounds of the present disclosure include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers that can be treated using one or more compounds of the present disclosure include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. In some aspects, compositions of the present disclosure may be used to treat a cancer selected from the group consisting of a hematologic cancer of the present disclosure or a hematologic cell proliferative disorder of the present disclosure, or used to identify suitable candidates for such purposes. A hematologic cancer of the present disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. In some aspects, compositions of the present disclosure may be used to treat lung cancer or cell proliferative disorders of the lung, or used to identify suitable candidates for such purposes. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

‘A “cell proliferative disorder of the colon” is a cell proliferative disorder involving cells of the colon. Preferably, the cell proliferative disorder of the colon is colon cancer. In some aspects, compositions of the present disclosure may be used to treat colon cancer or cell proliferative disorders of the colon, or used to identify suitable candidates for such purposes. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the present disclosure may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of TO or Tis; and where the regional lymph nodes (N) have been assigned a stage of NO; and where distant metastasis (M) has been assigned a stage of M0.

The cell proliferative disorder of the breast can be breast cancer. In some aspects, compositions of the present disclosure may be used to treat breast cancer, or used to identify suitable candidates for such purposes. Breast cancer may include all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphocytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructural heterogeneity (e.g., mixed cell types).

Compounds of the present disclosure can be used to treat breast cancer, or used to identify suitable candidates for such purposes. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the present disclosure may be used to treat or prevent a cell proliferative disorder of the breast, or to treat or prevent breast cancer, in a subject having an increased risk of developing breast cancer relative to the population at large, or used to identify suitable candidates for such purposes. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can be histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

In some embodiments, provided herein is a method of treating breast cancer comprising administering to a subject in need thereof an effective amount of a crystalline form of the hydrobromide of Compound I.

In some embodiments, provided herein is a method of treating breast cancer comprising administering to a subject in need thereof an effective amount of Polymorph A.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, NO, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol-), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the present disclosure (i.e., a crystalline form of the hydrobromide of Compound I, as well as Polymorph A) that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present disclosure. The biological or medical response can be the treatment of cancer. The biological or medical response can be treatment or prevention of a cell proliferative disorder. The biological response or effect can also include a change in cell proliferation or growth that occurs in vitro or in an animal model, as well as other biological changes that are observable in vitro. In vitro or in vivo biological assays can include, but are not limited to, enzymatic activity assays, electrophoretic mobility shift assays, reporter gene assays, in vitro cell viability assays, and the assays described herein.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present disclosure (i.e., a crystalline form of the hydrobromide of Compound I, as well as Polymorph A) to a subject in need of treatment of cancer. Monotherapy may be contrasted with combination therapy, in which a combination of multiple active compounds is administered, preferably with each component of the combination present in a therapeutically effective amount. In some aspects, monotherapy with a compound of the present disclosure is more effective than combination therapy in inducing a desired biological effect.

As used herein, “treating” or “treat” describes the management and care of a patient for the purpose of combating a disease, condition, or disorder and includes the administration of a compound of the present disclosure (i.e., a crystalline form of the hydrobromide of Compound I, as well as Polymorph A) to alleviate the symptoms or complications of a disease, condition or disorder, or to eliminate the disease, condition or disorder. The term “treat” can also include treatment of a cell in vitro or an animal model.

A compound of the present disclosure (i.e., a crystalline form of the hydrobromide of Compound I, as well as Polymorph A) can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In some aspects of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the disclosure.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present disclosure. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present disclosure. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound.

A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 60%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 60%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 60%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 60%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 60%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the present disclosure (i.e., a crystalline form of the hydrobromide of Compound I, as well as Polymorph A) acts selectively on a cancer or precancerous cell but not on a normal cell. Preferably, a compound of the present disclosure acts selectively to modulate one molecular target (e.g., a target protein methyltransferase) but does not significantly modulate another molecular target (e.g., a non-target protein methyltransferase). The disclosure also provides a method for selectively inhibiting the activity of an enzyme, such as a protein methyltransferase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the present disclosure can modulate the activity of a molecular target (e.g., a target protein methyltransferase). Modulating refers to stimulating or inhibiting an activity of a molecular target. Preferably, a compound of the present disclosure modulates the activity of a molecular target if it stimulates or inhibits the activity of the molecular target by at least 2-fold relative to the activity of the molecular target under the same conditions but lacking only the presence of said compound. More preferably, a compound of the present disclosure modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A compound of the present disclosure (i.e., a crystalline form of the hydrobromide of Compound I, as well as Polymorph A) does not significantly modulate the activity of a molecular target if the addition of the compound does not stimulate or inhibit the activity of the molecular target by greater than 10% relative to the activity of the molecular target under the same conditions but lacking only the presence of said compound.

As used herein, the term “isozyme selective” means preferential inhibition or stimulation of a first isoform of an enzyme in comparison to a second isoform of an enzyme (e.g., preferential inhibition or stimulation of a protein methyltransferase isozyme alpha in comparison to a protein methyltransferase isozyme beta). Preferably, a compound of the present disclosure demonstrates a minimum of a fourfold differential, preferably a tenfold differential, more preferably a fifty fold differential, in the dosage required to achieve a biological effect. Preferably, a compound of the present disclosure demonstrates this differential across the range of inhibition, and the differential is exemplified at the IC₅₀, i.e., a 50% inhibition, for a molecular target of interest.

Administering a compound of the present disclosure to a cell or a subject in need thereof can result in modulation (i.e., stimulation or inhibition) of an activity of a protein methyltransferase of interest.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a compound of the present disclosure is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present disclosure can induce or activate cell death selectively in cancer cells. Administering to a subject in need thereof a compound of the present disclosure can induce or activate cell death selectively in cancer cells. Contacting a cell with a compound of the present disclosure can induce cell death selectively in one or more cells affected by a cell proliferative disorder. Preferably, administering to a subject in need thereof a compound of the present disclosure induces cell death selectively in one or more cells affected by a cell proliferative disorder.

In some embodiments, the present disclosure relates to a method of treating or preventing cancer (e.g, the course of which can be influenced by modulating EZH2-mediated protein methylation) by administering a compound of the present disclosure (i.e., a crystalline form of the hydrobromide of Compound I, as well as Polymorph A) to a subject in need thereof, where administration of the compound of the present disclosure results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose. The present disclosure also relates to a method used to identify suitable candidates for treating or preventing cancer.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

### Examples

### Materials and Methods

### Powder X-Ray Diffraction

PXRD for all samples was taken on a Rigaku MultiFlex (Target: Cu; Tube voltage: 40 kV; Tube current: 30 mA).

### Differential Scanning Calorimetry

DSC for all samples was taken on a Mettler-Toledo DSC 1/700 (Run conditions: Initial temperature 35° C., Final temp 325° C., Heating rate 30° C./min).

### X-Ray Crystallography

A colorless plate crystal with dimensions 0.28×0.22×0.06 mm was mounted on a Nylon loop using very small amount of paratone oil. Data were collected using a Bruker CCD (charge coupled device) based diffractometer equipped with an Oxford Cryostream low-temperature apparatus operating at 173 K. Data were measured using omega and phi scans of 0.5° per frame for 45 s. The total number of images was based on results from the program COSMO where redundancy was expected to be 4.0 and completeness to 100% out to 0.83 Å. Cell parameters were retrieved using APEX II software and refined using SAINT on all observed reflections. Data reduction was performed using the SAINT software which corrects for Lp. Scaling and absorption corrections were applied using SADABS multi-scan technique, supplied by George Sheldrick. The structures are solved by the direct method using the SHELXS-97 program and refined by least squares method on F², SHELXL-97, which are incorporated in SHELXTL-PC V 6.10.

All non-hydrogen atoms are refined anisotropically. Hydrogens were calculated by geometrical methods and refined as a riding model. The crystal used for the diffraction study showed no decomposition during data collection. All drawings are done at 50% ellipsoids.

### Dynamic Vapor Sorption

Dynamic Vapor Sorption (DVS) was measured on a VTI Model SGA-100 system. Measurement method: The relative humidity (RH) was changed in a controlled fashion, in 5% steps from 5.0% to 95.0% then back to 5.0% using the gravimetric vapor sorption system, and the weight percentage change (wt. %) of the sample at each stage was measured.

### HPLC

HPLC was conducted on an Agilent 1200 HPLC quaternary pump, low pressure mixing, with an in-line degasser. Analytical method conditions: 8 μL sample (20 mg of ER-581982-06 diluted with 50 mL of a methanol to provide approximately 0.4 mg/mL solution) was injected onto a Agilent Zorbax Eclipse XDB-C18 (4.6×150 mm, 3.5 μm), Chromatography conditions: mobile phase A, water with 5 mM ammonium formate; mobile phase B, 5 mM ammonium formate in 50/45/5 acetonitrile/methanol/water; flow rate, 1.5 ml/min.; gradient: isocratic at 10% B from 0 to 3 min; linear increase to 70% B from 3 to 7 min; isocratic at 70% B from 7 to 12 min; linear increase to 100% B from 12 to 15 min isocratic at 100% B from 15 to 20 min; column temperature, 35° C.; detection, UV 230 nm. Approximate retention time of Compound I=10.7 min.

ssNMR

Because the XRPD method is not suitable to assess amorphous content, solid-state NMR (ss-NMR) was used to determine the amorphous content in drug substance batches. The limit of quantitation (LOQ) for this determination is approximately 5% w/w.

### Laser Diffraction

The drug substance particle size was evaluated by laser diffraction in an ethyl acetate dispersant (wet method). This method allows for measurement of the size distribution of primary particles without interference from potential agglomeration.

### Example 1: Synthesis of Compound I Hydrobromide

### Method Development

The following example describes a non-limiting embodiment of a synthesis of the disclosure. This non-limiting method was developed to accommodate scale-up, improve overall robustness, and increase quality of the resulting drug substance. For example, the exemplary method described in this example yielded high purity drug substance.

### I. Acidification Step

### Solvent Studies

Previous methods for the synthesis of Compound I hydrobromide generally utilized ethanol/water as a solvent for Compound I. In order to maintain a homogeneous ethanol/water solution for the HBr acidification, reaction temperatures of 65-70° C. were generally utilized. Under these conditions N-dealkylation decomposition was observed. In this non-limiting example, ethanol/water was replaced with ethanol/toluene as a solvent for Compound I. This allowed for a lowering of the reaction temperature to 25-35° C., which inhibited the pathway for N-dealkylation of Compound I.

### HBr Charge

The impact of excess HBr charges was investigated. In some embodiments, the results indicated that overcharging HBr showed significant effects on product quality, in addition to having increased bromide content. In addition, a high HBr charge can lead to the increased occurrence of dealkylation products. In some embodiments, the HBr charge was set to 0.985 mol eq.

II. Recrystallization step

Without wishing to be bound by theory, a recrystallization step was utilized to purge residual solvents (e.g., ethyl acetate, ethanol, toluene, etc.) from the drug substance, and to maintain consistency of solid-state properties. This resulted in residual solvent, including ethyl acetate, ethanol and toluene, at acceptable levels. Moreover, solid-state properties (form and particle size) were consistent with prior batches utilizing ethanol/water as a solvent for Compound I in the acidification step. However, elevated levels of ethanol and ethyl acetate were observed during the drying step following the recrystallization, resulting in extended drying times which did not significantly reduce the residual solvent levels. Further drying in a vacuum oven at elevated temperatures confirmed that further drying did not reduce ethanol and ethyl acetate levels. Without wishing to be bound by theory, this was taken as an indication that residual solvents were trapped/entrained within the crystalline particles of Compound I hydrobromide and it was postulated that the residual solvents may have been associated with the recrystallization process, rather than the drying process. Hence, a series of studies was initiated to better understand the recrystallization process. The studies are described below as cooling rate studies, particle size control studies, and solid-state form control studies. The parameters studied included cooling rate (or cool-down time), water content, isolation temperature, seed size, and seed quantity.

As a result of the studies the initial recrystallization procedure was altered in a variety of ways, including, but not limited to the following, which are described in more detail below: (1) the cool-down time in step b-2) was increased (2) the water content was decreased (3) the isolation temperature was increased (4) the seed size was decreased by micronization; (5) the seed quantity was increased; and/or (6) an isothermal hold time was implemented after seeding (e.g., step b-1). As described below, the revised conditions provided a robust crystallization process with adequate form control, producing drug substance with low levels of residual solvents and a suitable particle size.

### Cooling Rate Studies

Studies were performed to evaluate the controlled crystallization pathway. A cool-down time of no more than 10 h (e.g. in step b-2) after a seed hold time of at least 2 h (e.g. in step b-1) provided a robust crystallization process. This reduced the residual solvent content by a factor 10 for ethanol compared to previous experiments where the cool-down time was 3 h. Throughout development of the methods of the disclosure, residual ethyl acetate was reduced from around 2,700 ppm to less than 100 ppm and toluene was reduced from over 80 ppm in previous experiments to less than or equal to 20 ppm.

### Particle Size Control Studies

Without wishing to be bound by theory, implementing the longer cool-down time described above increased the size of the resulting drug substance particle size. Hence, further development focused on reducing the drug substance particle size by: (1) reducing the particle size of the seeds by micronization and/or (2) increasing the seed load. Studies revealed that the combination of micronized seeds (e.g., ≤5 m) and an increase in the seed load (e.g., 2.0 wt. %) resulted in a D90 particle size of approximately 30 μm, i.e., within a targeted range of 15-50 μm proposed. Moreover, the distribution obtained was narrow, with greater than 90% of the particles having a size (diameter) between 6 μm and 40 μm.

### Solid-State Form Control Studies

The robustness of the non-limiting methods described in this example with respect to form control was studied. Compound I hydrobromide is a polymorphic molecule that can exist as several stable forms. Besides Polymorph A, there are nine alternative forms that are associated with crystallization solvents of the methods of the disclosure. A quantitative X-ray Powder Diffraction (XRPD) method was developed to detect the presence of these alternative forms in the drug substance. In addition, solid-state NMR (ss-NMR) was used to detect the presence of amorphous content. Of the forms, Polymorph B is one of the forms at the highest risk to be present in addition to Polymorph A. Solubility studies were performed for both Polymorph A and Polymorph B which showed that the Polymorph A and Polymorph B solubility curves cross at a low temperature, indicated as the thermodynamic stability barrier. The results also indicated that the barrier temperature decreases with decreasing water content.

To determine the thermodynamically most stable form as a function of temperature and water content in the solvent, competitive slurry experiments were performed. Several slurries were prepared, varying the water content and the temperature. The slurries used a 50/50% (w/w) mixture of Polymorph A and Polymorph B, and were slurried for several days to find the most stable form. The results indicated that Polymorph A is the stable form at higher temperatures and lower water content, while Polymorph B is the stable form at lower temperature and higher water content. As a result, the isolation temperature (e.g. in step b-2) was increased (e.g., from 15° C. to 22° C.) and the water content of the solvent system during the cooling crystallization was reduced (e.g., from 9.7% (v/v) to 9.0% (v/v)).

Furthermore, in some experiments, a temperature cycle was inserted at the end of the recrystallization step, after addition of the ethyl acetate anti-solvent: The slurry was heated to a temperature between about 43° C. to about 57° C. and stirred for at least 1 h. Then the slurry was cooled to a temperature between about 13° C. and about 18° C. for about an hour. Lastly, the slurry was stirred for at least another 1 h. This procedure was shown to aid in converting any potential Polymorph B to Polymorph A, providing further assurance of form control during recrystallization.

Particle size distribution profiles for batches made by the methods of the disclosure were evaluated by laser diffraction. A 90% cumulative particle diameter was consistent across development of the recrystallization step (e.g., 27.3 μm on average for earlier batches, 31.7 μm on average for batches made by methods of the disclosure). However, the particle size distribution was narrower for the batches made by the methods of the disclosure.

### Example 2: Assessment of Hydrobromide of Compound I and Polymorph A

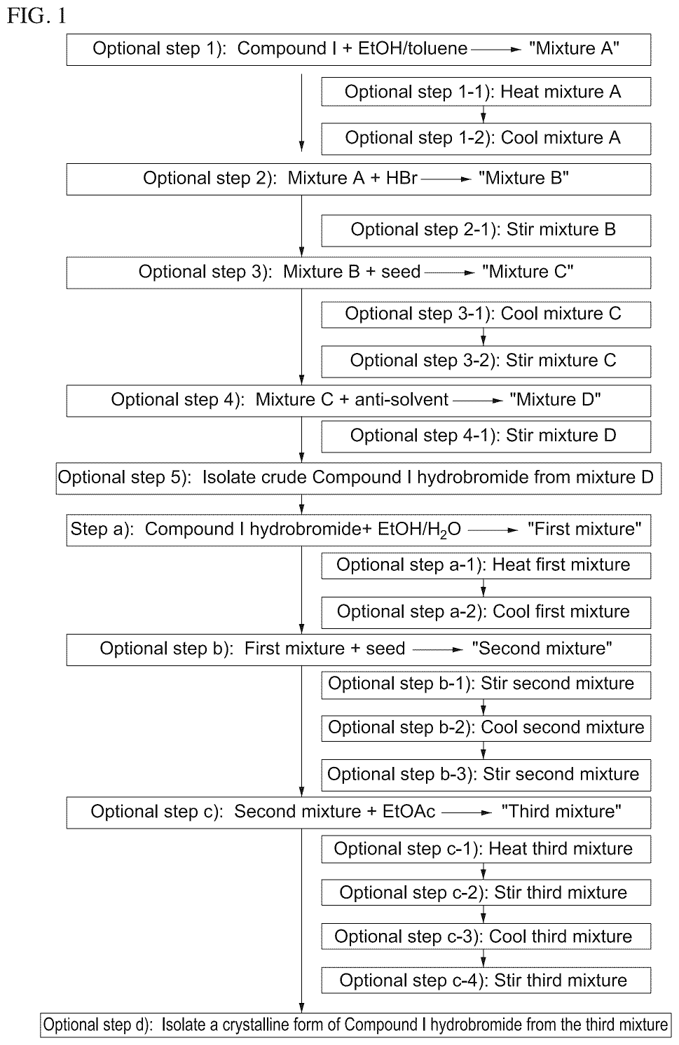

The X-ray powder diffraction pattern of Polymorph A (monohydrobromide) is shown e.g., in FIG. 1 in U.S. Pat. No. 9,394,283, incorporated by reference herein in its entirety). Table 1, below, lists the most significant peaks.

TABLE 1
Peaks (Degrees 2-theta)

3.9
10.1
14.3
17.5
18.7
20.6
20.9
21.8
22.0
23.3
23.6

### Exemplary Embodiments

Embodiment 1. A method of making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

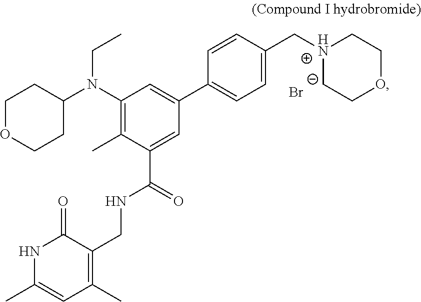

comprising:
step a) mixing Compound I hydrobromide, ethanol, and water, wherein the vol/vol ratio of ethanol:water is from about 92:8 to about 87:13, to form a first mixture.
   Embodiment 2. The method of embodiment 1, wherein the vol/vol ratio of ethanol:water is from about 91.5:8.5 to about 87.5:12.5.
   Embodiment 3. The method of embodiment 1, wherein the vol/vol ratio of ethanol:water is about 91:9.
   Embodiment 4. The method of any one of the preceding embodiments, wherein the vol/vol ratio of ethanol:water in step a) is about 92:8, about 91.5:8.5, about 91:9, about 90.5:9.5, about 90:10, about 89.5:10.5, about 89:11, about 88.5:11.5, about 88:12, or about 87.5:12.5.
   Embodiment 5. The method of any one of the preceding embodiments, further comprising after step a): step b) adding a seed to the first mixture to form a second mixture.
   Embodiment 6. A method of making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

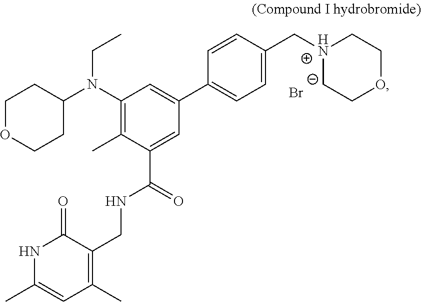

comprising:
step a′) mixing Compound I hydrobromide, ethanol, and water to form a first mixture; and
step b) adding a seed to the first mixture to form a second mixture, wherein step b) is after step a′).
   Embodiment 7. The method of any one of the preceding embodiments, wherein the vol/vol ratio of ethanol:water in step a′) is about 92:8, about 91.5:8.5, about 91:9, about 90.5:9.5, about 90:10, about 89.5:10.5, about 89:11, about 88.5:11.5, about 88:12, or about 87.5:12.5.
   Embodiment 8. The method of any one of the preceding embodiments, wherein the vol/vol ratio of ethanol:water in step a′) is about 91.3:8.7, about 91.2:8.8, about 91.1:8.9, about 91.0:9.0, about 90.9:9.1, about 90.8:9.2, about 90.7:9.3.
   Embodiment 9. A method of making a crystalline form of Compound I hydrobromide comprising:
step a) mixing Compound I hydrobromide, ethanol, and water, wherein the vol/vol ratio of ethanol:water is about 91:9, to form a first mixture; and
step b) adding a seed to the first mixture to form a second mixture, wherein step b) is after step a).
   Embodiment 10. The method of any one of the preceding embodiments, further comprising after step a) and before step b): step a-1) heating the first mixture.
   Embodiment 11. The method of any one of the preceding embodiments, further comprising after step a′) and before step b): step a-1) heating the first mixture.
   Embodiment 12. The method of any one of the preceding embodiments, further comprising after step a-1): step a-2) cooling the first mixture.
   Embodiment 13. The method of any one of the preceding embodiments, further comprising after step b): step b-1) stirring the second mixture.
   Embodiment 14. The method of any one of the preceding embodiments, further comprising after step b-1): step b-2) cooling the second mixture.
   Embodiment 15. The method of any one of the preceding embodiments, further comprising after step b-2): step b-3) stirring the second mixture.
   Embodiment 16. The method of any one of the preceding embodiments, further comprising after step b-3): step c) adding an anti-solvent to the second mixture to form a third mixture.
   Embodiment 17. The method of any one of the preceding embodiments, further comprising after step b-3): step c) adding ethyl acetate to the second mixture to form a third mixture.
   Embodiment 18. The method of any one of the preceding embodiments, further comprising after step b-2): step c) adding an anti-solvent to the second mixture to form a third mixture.
   Embodiment 19. The method of any one of the preceding embodiments, further comprising after step c): step c-1) heating the third mixture.
   Embodiment 20. The method of any one of the preceding embodiments, further comprising after step c-1): step c-2) stirring the third mixture.
   Embodiment 21. The method of any one of the preceding embodiments, further comprising after step c-2): step c-3) cooling the third mixture.
   Embodiment 22. The method of any one of the preceding embodiments, further comprising after step c-3): step c-4) stirring the third mixture.
   Embodiment 23. The method of any one of the preceding embodiments, wherein in step c-1) the third mixture is heated to a temperature of from about 45° C. to about 55° C. or from about 47° C. to about 53° C.
   Embodiment 24. The method of any one of the preceding embodiments, wherein in step c-1) the third mixture is heated to a temperature of about 47° C., about 48° C., about 49° C., about 50° C., about 51° C., about 52° C., or about 53° C.
   Embodiment 25. The method of any one of the preceding embodiments, wherein in step c-2) the third mixture is stirred for at least about 1 h.
   Embodiment 26. The method of any one of the preceding embodiments, wherein in step c-2) the third mixture is stirred for about 1 h, about 2 h, about 3 h, about 4 h, or about 5 h or more.
   Embodiment 27. The method of any one of the preceding embodiments, wherein in step c-3) the third mixture is cooled to a temperature of from about 10° C. to about 40° C., from about 10° C. to about 35° C., from about 18° C. to about 35° C., or from about 10° C. to about 20° C.
   Embodiment 28. The method of any one of the preceding embodiments, wherein in step c-4) the third mixture is stirred for about 1 h, about 2 h, about 3 h, about 4 h, or about 5 h or more.
   Embodiment 29. The method of any one of the preceding embodiments, further comprising after step c): step d) isolating the crystalline form of Compound I hydrobromide from the third mixture.
   Embodiment 30. The method of any one of the preceding embodiments, wherein in step d) the crystalline form of Compound I hydrobromide is isolated from the third mixture by filtration.
   Embodiment 31. A method of making a crystalline form of Compound I hydrobromide comprising:
step a) mixing Compound I hydrobromide, ethanol, and water, wherein the vol/vol ratio of ethanol and water is about 91:9, to form a first mixture; 25:75 to about 45:55; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).
   Embodiment 32. A method of making a crystalline form of Compound I hydrobromide consisting essentially of:
step a′) mixing Compound I hydrobromide, ethanol, and water to form a first mixture; and
step b) adding a seed to the first mixture to form a second mixture; wherein step b) is after step a′).
   Embodiment 33. A method of making a crystalline form of Compound I hydrobromide consisting essentially of:
step a) mixing Compound I hydrobromide, ethanol, and water, wherein the vol/vol ratio of ethanol and water is about 91:9, to form a first mixture; and
step b) adding a seed to the first mixture to form a second mixture; wherein step b) is after step a).
   Embodiment 34. A method of making a crystalline form of Compound I hydrobromide consisting essentially of:
step a) mixing Compound I hydrobromide, ethanol, and water, wherein the vol/vol ratio of ethanol and water is about 91:9, to form a first mixture;
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).
   Embodiment 35. A method of making a crystalline form of Compound I hydrobromide consisting essentially of:
step a′) mixing Compound I hydrobromide, ethanol, and water to form a first mixture;
step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).
   Embodiment 36. A method of making a crystalline form of Compound I hydrobromide consisting essentially of:
step a) mixing Compound I hydrobromide, ethanol, and water, wherein the vol/vol ratio of ethanol and water is about 91:9, to form a first mixture;
step a-1) heating the first mixture; wherein step a-1) is after step a);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).
   Embodiment 37. A method of making a crystalline form of Compound I hydrobromide consisting essentially of:
step a′) mixing Compound I hydrobromide, ethanol, and water to form a first mixture;
step a-1) heating the first mixture; wherein step a-1) is after step a′);
step a-2) cooling the first mixture; wherein step a-2) is after step a-1);
step b) adding a seed to the first mixture to form a second mixture; wherein step b) is after step a-2);
step b-1) stirring the second mixture; wherein step b-1) is after step b);
step b-2) cooling the second mixture; wherein step b-2) is after step b-1);
step b-3) stirring the second mixture; wherein step b-3) is after step b-2);
step c) adding an anti-solvent to the second mixture to form a third mixture; wherein step c) is after step b-3); and
step d) isolating the crystalline form of Compound I hydrobromide from the third mixture; wherein step d) is after step c).
   Embodiment 38. The method of any one of the preceding embodiments, wherein in step b) the amount of seed in the second mixture is from about 1.0 wt. % to about 3.0 wt. % relative to the crystalline form of Compound I hydrobromide.
   Embodiment 39. The method of any one of the preceding embodiments, wherein in step b) the amount of seed in the second mixture is from about 1.96 wt. % to about 2.04 wt. % relative to the crystalline form of Compound I hydrobromide.
   Embodiment 40. The method of any one of the preceding embodiments, wherein in step b) the amount of seed in the second mixture is about 2.0 wt. % relative to the crystalline form of Compound I hydrobromide.
   Embodiment 41. The method of any one of the preceding embodiments, wherein the 90% cumulative particle diameter in particle size distribution of the seed in step b) is 6 μm or less.
   Embodiment 42. The method of any one of the preceding embodiments, wherein the 90% cumulative particle diameter in particle size distribution of the seed in step b) is 5 μm or less.
   Embodiment 43. The method of any one of the preceding embodiments, wherein the 90% cumulative particle diameter in particle size distribution of the seed in step b) is from about 4 μm to about 6 μm.
   Embodiment 44. The method of any one of the preceding embodiments, wherein the seed in step b) is Compound I hydrobromide.

Embodiment 45. The method of any one of the preceding embodiments, wherein the seed in step b) is amorphous Compound I hydrobromide.

Embodiment 46. The method of any one of the preceding embodiments, wherein the seed in step b) is a crystalline form of Compound I hydrobromide.

Embodiment 47. The method of any one of the preceding embodiments, wherein the seed in step b) is Polymorph A of Compound I hydrobromide.

Embodiment 48. The method of any one of the preceding embodiments, wherein the seed in step b) exhibits an X-ray powder diffraction pattern having one or two characteristic peaks expressed in degrees 2-theta, selected from the group consisting of 17.5+/−0.3 and 22.0+/−0.3.

Embodiment 49. The method of any one of the preceding embodiments, wherein in step a-1) the first mixture is heated to a temperature of from about 70° C. to about 75° C.

Embodiment 50. The method of any one of the preceding embodiments, wherein in step a-2) the first mixture is cooled to a temperature of from about 45° C. to about 55° C.

Embodiment 51. The method of any one of the preceding embodiments, wherein in step a-2) the first mixture is cooled to a temperature of from about 50° C. to about 55° C.

Embodiment 52. The method of any one of the preceding embodiments, wherein in step b-2) the second mixture is cooled at a cooling rate of from about 2° C./h to about 9° C./h.

Embodiment 53. The method of any one of the preceding embodiments, wherein in step b-2) the second mixture is cooled at a cooling rate of about 3° C./h.

Embodiment 54. The method of any one of the preceding embodiments, wherein in step b-2) the second mixture is cooled to a temperature of from about 18° C. to about 30° C.

Embodiment 55. The method of any one of the preceding embodiments, wherein in step b-2) the second mixture is cooled to a temperature of from about 20° C. to about 25° C.

Embodiment 56. The method of any one of the preceding embodiments, wherein in step b-2) the second mixture is cooled to a temperature of about 22° C.

Embodiment 57. The method of any one of the preceding embodiments, wherein in step c) the anti-solvent is added to the third mixture the over a time period of from about 1 h to about 5 h.

Embodiment 58. The method of any one of the preceding embodiments, wherein in step c) the anti-solvent is added to the third mixture over a time period of from about 3 h to about 5 h. Embodiment 59. The method of any one of the preceding embodiments, wherein in step c), the entire amount of the anti-solvent is added at once.

Embodiment 60. The method of any one of the preceding embodiments, wherein in step c) the anti-solvent is added in amount of from about 5 volumes to about 15 volumes.

Embodiment 61. The method of any one of the preceding embodiments, wherein in step c) the anti-solvent is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

Embodiment 62. The method of any one of the preceding embodiments, wherein in step c) the anti-solvent is added in an amount of about 9 volumes.

Embodiment 63. The method of any one of the preceding embodiments, wherein in step c) the anti-solvent is added until crystalline particles of Compound I hydrobromide form.

Embodiment 64. The method of any one of the preceding embodiments, wherein the anti-solvent in step c) is selected from ethyl acetate, methyl tert-butyl ether, tetrahydrofuran, and acetone.

Embodiment 65. The method of any one of the preceding embodiments, wherein the anti-solvent in step c) is ethyl acetate.

Embodiment 66. The method of any one of the preceding embodiments, wherein in step c) ethyl acetate is added to the third mixture the over a time period of from about 1 h to about 5 h.

Embodiment 67. The method of any one of the preceding embodiments, wherein step c) ethyl acetate is added to the third mixture over a time period of from about 3 h to about 5 h.

Embodiment 68. The method of any one of the preceding embodiments, wherein in step c), the entire amount of ethyl acetate is added at once.

Embodiment 69. The method of any one of the preceding embodiments, wherein in step c) ethyl acetate is added in amount of from about 5 volumes to about 15 volumes.

Embodiment 70. The method of any one of the preceding embodiments, wherein in step c) ethyl acetate is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.

Embodiment 71. The method of any one of the preceding embodiments, wherein in step c) ethyl acetate is added in an amount of about 9 volumes.

Embodiment 72. The method of any one of the preceding embodiments, wherein in step c) ethyl acetate is added until crystalline particles of the crystalline form of Compound I hydrobromide form.

Embodiment 73. The method of any one of the preceding embodiments, wherein the method comprises before step a):
step 1) mixing Compound I, ethanol, and toluene to form mixture A; and after step 1):
step 2) adding hydrobromic acid to mixture A to form a mixture B, wherein Compound I hydrobromide is formed.

Embodiment 74. The method of any one of the preceding embodiments, wherein the method comprises before step a):
step 1) mixing Compound I, a first solvent, and a second solvent to form mixture A; and after step 1):
step 2) adding hydrobromic acid to mixture A to form a mixture B, wherein Compound I hydrobromide is formed.

Embodiment 75. A method of making a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

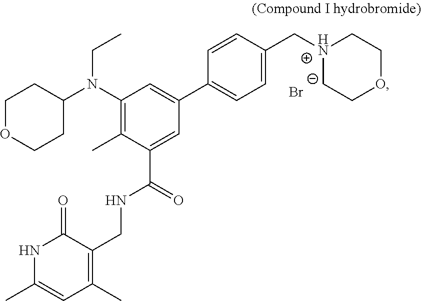

wherein the method comprises:
step 1) mixing Compound I, a first solvent, and a second solvent to form mixture A; and after step 1):
step 2) adding hydrobromic acid to mixture A to form a mixture B, wherein Compound I hydrobromide is formed.
   Embodiment 76. The method of any one of the preceding embodiments, wherein the method comprises before step a):
step 1) mixing Compound I, a first solvent, and a second solvent to form mixture A; and after step 1):
step 2) adding hydrobromic acid to mixture A to form a mixture B, wherein Compound I hydrobromide is formed.
   Embodiment 77. The method of any one of the preceding embodiments, wherein the method further comprises after step 3):
step 4) adding an anti-solvent to mixture C to form mixture D; and after step 4):
step 5) isolating crude Compound I hydrobromide from mixture D.
   Embodiment 78. The method of any one of the preceding embodiments, wherein the method further comprises after step 5):
step a) mixing Compound I hydrobromide, ethanol, and water, wherein the vol/vol ratio of ethanol:water is from about 92:8 to about 87:13, to form a first mixture.
   Embodiment 79. The method of any one of the preceding embodiments, wherein the method further comprises after step 5):
step a) mixing Compound I hydrobromide and a third solvent to form a first mixture.
   Embodiment 80. The method of any one of the preceding embodiments, wherein the method further comprises after step a):
step b) adding a seed to the first mixture to form a second mixture.
   Embodiment 81. The method of any one of the wherein the third solvent comprises methanol, ethanol, water, propanol, tetrahydrofuran, acetone, acetonitrile, and mixtures thereof.
   Embodiment 82. The method of any one of the wherein the third solvent comprises ethanol and water.
   Embodiment 83. The method of any one of the preceding embodiments, wherein the method further comprises after step a):
step b): adding a seed to the first mixture to form a second mixture.
   Embodiment 84. The method of any one of the preceding embodiments, wherein in step 2) hydrobromic acid is added to mixture B at a temperature of from about 10° C. to about 50° C.
   Embodiment 85. The method of any one of the preceding embodiments, wherein in step 2) hydrobromic acid is added to mixture B at a temperature of about 10° C., about 15° C., about 20° C., about 25° C., about 30° C., about 35° C., about 40° C., about 45° C., or about 50° C.
   Embodiment 86. The method of any one of the preceding embodiments, wherein the first solvent is selected from the group consisting of water, ethanol, methanol, propanol, benzyl alcohol, tetrahydrofuran, acetone, acetonitrile, acetic acid, ethylene glycol, and mixtures thereof.
   Embodiment 87. The method of any one of the preceding embodiments, wherein the first solvent is selected from the group consisting 1-butanol, 2-butanol, 3-methyl-1-butanol, 2-methyl-1-propanol, 1-pentanol, 1-propanol, and 2-propanol.
   Embodiment 88. The method of any one of the preceding embodiments, wherein the first solvent is or comprises ethanol.
   Embodiment 89. The method of any one of the preceding embodiments, wherein the second solvent is or comprises tetralin or 1,1,2-trichloroethene.
   Embodiment 90. The method of any one of the preceding embodiments, wherein the second solvent is or comprises an aromatic compound.
   Embodiment 91. The method of any one of the preceding embodiments, wherein the second solvent is selected from toluene, benzene, ethyl benzene, and xylene.
   Embodiment 92. The method of any one of the preceding embodiments, wherein the second solvent is or comprises toluene.
   Embodiment 93. The method of any one of the preceding embodiments, wherein the first solvent is or comprises ethanol.
   Embodiment 94. The method of any one of the preceding embodiments, wherein the method further comprises after step 2): step 3) adding a seed to mixture B to form mixture C.
   Embodiment 95. The method of any one of the preceding embodiments, wherein the method comprises before step a′):
step 1) mixing Compound I, ethanol, and toluene to form mixture A; and
step 2) adding hydrobromic acid to mixture A to form a mixture B, wherein Compound I hydrobromide is formed; and wherein step 2) is after step 1).
   Embodiment 96. The method of any one of the preceding embodiments, wherein the method comprises before step a):
step 1) mixing Compound I, ethanol, and toluene to form mixture A;
step 2) adding hydrobromic acid to mixture A to form a mixture B, wherein Compound I hydrobromide is formed; and wherein step 2) is after step 1);
step 3) adding a seed to mixture B to form mixture C; wherein step 3) is after step 2);
step 4) adding an anti-solvent to mixture C to form mixture D; wherein step 4) is after step 3); and
step 5) isolating crude Compound I hydrobromide from mixture D; wherein step 5) is after step 4).
   Embodiment 97. The method of any one of the preceding embodiments, wherein the method comprises before step a′):
step 1) mixing Compound I, ethanol, and toluene to form mixture A;
step 2) adding hydrobromic acid to mixture A to form a mixture B, wherein Compound I hydrobromide is formed; and wherein step 2) is after step 1);
step 3) adding a seed to mixture B to form mixture C; wherein step 3) is after step 2);
step 4) adding an anti-solvent to mixture C to form mixture D; wherein step 4) is after step 3); and
step 5) isolating crude Compound I hydrobromide from mixture D; wherein step 5) is after step 4).
   Embodiment 98. The method of any one of the preceding embodiments, wherein the method comprises before step a):
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form a mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1);
step 3-1) cooling mixture C; wherein step 3-1) is after step 3);
step 3-2) stirring mixture C for ≥2 h; wherein step 3-2) is after step 3-1);
step 4) adding an anti-solvent to mixture C to form mixture D; wherein step 4) is after step 3-2);
step 4-1) stirring mixture D for ≥4 h; wherein step 4-1) is after step 4); and
step 5) isolating crude Compound I hydrobromide from mixture D; wherein step 5) is after step 4).
   Embodiment 99. The method of any one of the preceding embodiments, wherein the method comprises before step a′):
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form a mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C for ≥2 h; wherein step 3-2) is after step 3-1);
step 4) adding an anti-solvent to mixture C to form mixture D; wherein step 4) is after step 3-2);
step 4-1) stirring mixture D for ≥4 h; wherein step 4-1) is after step 4); and
step 5) isolating crude Compound I hydrobromide from mixture D; wherein step 5) is after step 4).
   Embodiment 100. A method of making a crystalline form of Compound I hydrobromide consisting essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a) mixing Compound I hydrobromide, ethanol, and water, wherein the vol/vol ratio of ethanol and water is about 91:9, to form a first mixture; wherein step a) is after step 2); and
step b) adding a seed to the first mixture to form a second mixture; wherein step b) is after step a).
   Embodiment 101. A method of making a crystalline form of Compound I hydrobromide consisting essentially of:
step 1) mixing Compound I, ethanol, and toluene to form mixture A;
step 2) adding hydrobromic acid to mixture A to form mixture B, wherein Compound I hydrobromide is formed; and wherein step 2) is after step 1);
step a′) mixing Compound I hydrobromide, ethanol, and water; wherein step a′) is after step 2); and
step b) adding a seed to the first mixture to form a second mixture; wherein step b) is after step a′).
   Embodiment 102. A method of making a crystalline form of Compound I hydrobromide consisting essentially of:
step 1) mixing Compound I, ethanol, and toluene to form mixture A;
step 1-1) heating mixture A; wherein step 1-1) is after step 1);
step 1-2) cooling mixture A; wherein step 1-2) is after step 1-1);
step 2) adding hydrobromic acid to mixture A to form a mixture B, wherein Compound I hydrobromide is formed; and wherein step 2) is after step 1-2);
step 2-1) stirring mixture B; wherein step 2-1) is after step 2);
step 3) adding a seed to mixture B to form mixture C; wherein step 3) is after step 2-1); step 3-1) cooling mixture C; wherein step 3-1) is after step 3);
step 3-2) stirring mixture C; wherein step 3-2) is after step 3-1);
step 4) adding an anti-solvent to mixture C to form mixture D; wherein step 4) is after step 3-2);
step 4-1) stirring mixture D for ≥4 h; wherein step 4-1) is after step 4);
step 5) isolating crude Compound I hydrobromide from mixture D; wherein step 5) is after step 4);
step a) mixing Compound I hydrobromide, ethanol, and water, wherein the vol/vol ratio of ethanol and water is about 91:9, to form a first mixture; wherein step a) is after step 5);
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).
   Embodiment 103. A method of making a crystalline form of Compound I hydrobromide consisting essentially of:
step 1) mixing Compound I, ethanol, and toluene to form mixture A;
step 2) adding hydrobromic acid to mixture A to form a mixture B, wherein Compound I hydrobromide is formed; and wherein step 2) is after step 1);
step 3) adding a seed to mixture B to form mixture C; wherein step 3) is after step 2); step 4) adding an anti-solvent to mixture C to form mixture D; wherein step 4) is after step 3);
step 5) isolating crude Compound I hydrobromide from mixture D; wherein step 5) is after step 4);
step a) mixing Compound I hydrobromide, ethanol, and water, wherein the vol/vol ratio of ethanol and water is about 91:9, to form a first mixture; wherein step a) is after step 5);
step b) adding a seed to the first mixture to form a second mixture; wherein step b) is after step a);
step c) adding an anti-solvent to the second mixture to form a third mixture; wherein step c) is after step b); and
step d) isolating the crystalline form of Compound I hydrobromide from the third mixture; wherein step d) is after step c).
   Embodiment 104. A method of making a crystalline form of Compound I hydrobromide consisting essentially of:
step 1) mixing Compound I, ethanol, and toluene to form mixture A;
step 2) adding hydrobromic acid to mixture A to form a mixture B, wherein Compound I hydrobromide is formed; and wherein step 2) is after step 1);
step 3) adding a seed to mixture B to form mixture C; wherein step 3) is after step 2);
step 4) adding an anti-solvent to mixture C to form mixture D; wherein step 4) is after step 3);
step 5) isolating crude Compound I hydrobromide from mixture D; wherein step 5) is after step 4);
step a′) mixing Compound I hydrobromide, ethanol, and water to form a first mixture; wherein step a′) is after step 5);
step b) adding a seed to the first mixture to form a second mixture; wherein step b) is after step a′);
step c) adding an anti-solvent to the second mixture to form a third mixture; wherein step c) is after step b); and
step d) isolating the crystalline form of Compound I hydrobromide from the third mixture; wherein step d) is after step c).
   Embodiment 105. The method of any one of the preceding embodiments, wherein in step 1) the vol/vol ratio of ethanol:toluene in mixture A is about 25:75 to about 45:55.
   Embodiment 106. The method of any one of the preceding embodiments, wherein in step 1) the vol/vol ratio of ethanol:toluene in mixture A is about 25:75, about 30:70, about 35:65, about 40:60, or about 45:55.
   Embodiment 107. The method of any one of the preceding embodiments, wherein in step 1) the vol/vol ratio of ethanol:toluene in mixture A is about 40:60.
   Embodiment 108. The method of any one of the preceding embodiments, wherein in step 1-1), mixture A is heated to a temperature of from about 40° C. to about 80° C.
   Embodiment 109. The method of any one of the preceding embodiments, wherein in step 1-1), mixture A is heated to a temperature of from about 60° C. to about 70° C.
   Embodiment 110. The method of any one of the preceding embodiments, wherein in step 1-1), mixture A is heated to a temperature of about 40° C., about 45° C., about 50° C., about 55° C., about 60° C., about 65° C., about 70° C., about 75° C., or about 80° C.
   Embodiment 111. The method of any one of the preceding embodiments, wherein in step 1-2), the mixture is cooled to a temperature of from about 20° C. to about 40° C.
   Embodiment 112. The method of any one of the preceding embodiments, wherein in step 1-2), the mixture is cooled to a temperature of from about 25° C. to about 35° C.
   Embodiment 113. The method of any one of the preceding embodiments, wherein in step 1-2), the mixture is cooled to a temperature of about 20° C., about 25° C., about 30° C., about 35° C., or about 40° C.
   Embodiment 114. The method of any one of the preceding embodiments, wherein in step 1-2), the mixture is cooled to a temperature of 30° C.
   Embodiment 115. The method of any one of the preceding embodiments, wherein in step 2), hydrobromide is added to mixture B at a temperature of from about 20° C. to about 40° C.
   Embodiment 116. The method of any one of the preceding embodiments, wherein in step 2), hydrobromide is added to mixture B at a temperature of from about 25° C. to about 35° C.
   Embodiment 117. The method of any one of the preceding embodiments, wherein in step 2), hydrobromide is added to mixture B at a temperature of about 20° C., about 25° C., about 30° C., about 35° C., or about 40° C.
   Embodiment 118. The method of any one of the preceding embodiments, wherein in step 2), hydrobromide is added to mixture B at a temperature of about 30° C.
   Embodiment 119. The method of any one of the preceding embodiments, wherein in step 2) hydrobromic acid is added to mixture A in an amount of from about 0.9 mol eq. to about 1.1 mol eq. with respect to Compound I.
   Embodiment 120. The method of any one of the preceding embodiments, wherein in step 2) hydrobromic acid is added to mixture A in an amount of from about 0.95 mol eq. to about 1.05 mol eq. with respect to Compound I.
   Embodiment 121. The method of any one of the preceding embodiments, wherein, in step 2) hydrobromic acid is added to mixture A in an amount of from about 0.975 mol eq. to about 0.990 mol eq. with respect to Compound I.
   Embodiment 122. The method of any one of the preceding embodiments, wherein, in step 2) hydrobromic acid is added to mixture A in an amount of from about 0.975 mol eq. to about 0.995 mol eq. with respect to Compound I.
   Embodiment 123. The method of any one of the preceding embodiments, wherein in step 2) hydrobromic acid is added to mixture A in an amount of from about 0.98 mol eq. to about 1.00 mol eq. with respect to Compound I.
   Embodiment 124. The method of any one of the preceding embodiments, wherein in step 2) hydrobromic acid is added to mixture A in an amount of about 0.95 mol eq., about 0.96 mol eq., about 0.97 mol eq., about 0.98 mol eq., about 0.99 mol eq., about 1.00 mol eq., about 1.01 mol eq., about 1.02 mol eq., about 1.03 mol eq., about 1.04 mol eq., or about 1.05 mol eq. with respect to Compound I.
   Embodiment 125. The method of any one of the preceding embodiments, wherein in step 2) hydrobromic acid is added to mixture A in an amount of about 0.99 mol eq. with respect to Compound I.
   Embodiment 126. The method of any one of the preceding embodiments, wherein in step 2) hydrobromic acid is added in an amount of about 0.985 mol eq. with respect to Compound I.
   Embodiment 127. The method of any one of the preceding embodiments, wherein in step 3) the amount of seed in mixture B is from about 1.96 wt. % to about 2.04 wt. %.
   Embodiment 128. The method of any one of the preceding embodiments, wherein in step 3) the amount of seed in mixture B is about 1.96 wt. %, about 1.97 wt. %, about 1.98 wt. %, about 2.00 wt. %, about 2.01 wt. %, about 2.02 wt. %, about 2.03 wt. %, or about 2.04 wt. %.
   Embodiment 129. The method of any one of the preceding embodiments, wherein in step 3) the D90 particle size of the seed is 6 μm or less.
   Embodiment 130. The method of any one of the preceding embodiments, wherein in step 3) the D90 particle size of the seed is 5 μm or less.
   Embodiment 131. The method of any one of the preceding embodiments, wherein in step 3) the D90 particle size of the seed is from about 4 μm to about 6 μm.
   Embodiment 132. The method of any one of the preceding embodiments, wherein in step 3) the D90 particle size of the seed is about 3 μm, about 4 μm, about 5 μm, or about 6 μm.
   Embodiment 133. The method of any one of the preceding embodiments, wherein the seed in step 3) is Compound I hydrobromide.
   Embodiment 134. The method of any one of the preceding embodiments, wherein the seed in step 3) is amorphous Compound I hydrobromide.
   Embodiment 135. The method of any one of the preceding embodiments, wherein the seed in step 3) is a crystalline form of Compound I hydrobromide.
   Embodiment 136. The method of any one of the preceding embodiments, wherein the seed in step 3) is Polymorph A of Compound I hydrobromide.
   Embodiment 137. The method of any one of the preceding embodiments, wherein the seed in step 3) exhibits an X-ray powder diffraction pattern having one or two characteristic peaks expressed in degrees 2-theta, selected from the group consisting of 17.5+/−0.3 and 22.0+/−0.3.
   Embodiment 138. The method of any one of the preceding embodiments, wherein in step 3-1), the mixture is cooled to a temperature of from about 0° C. to about 20° C.
   Embodiment 139. The method of any one of the preceding embodiments, wherein in step 3-1), the mixture is cooled to a temperature of from about 5° C. to about 15° C.
   Embodiment 140. The method of any one of the preceding embodiments, wherein in step 3-1), the mixture is cooled to a temperature of about 5° C., about 6° C., about 7° C., about 8° C., about 9° C., about 10° C., about 11° C., about 12° C., about 13° C., about 14° C., or about 15° C.
   Embodiment 141. The method of any one of the preceding embodiments, wherein in step 4) the anti-solvent is added over a time period of from about 1 h to about 5 h.
   Embodiment 142. The method of any one of the preceding embodiments, wherein in step 4) the anti-solvent is added over a time period of from about 3 h to about 5 h.
   Embodiment 143. The method of any one of the preceding embodiments, wherein in step 4) the anti-solvent is added over a time period of about 1 h, about 2 h, about 3 h, about 4 h, or about 5 h.
   Embodiment 144. The method of any one of the preceding embodiments, wherein in step 4), the entire amount of the anti-solvent is added at once.
   Embodiment 145. The method of any one of the preceding embodiments, wherein in step 4) the anti-solvent is added in amount of from about 5 volumes to about 15 volumes.
   Embodiment 146. The method of any one of the preceding embodiments, wherein in step 4) the anti-solvent is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.
   Embodiment 147. The method of any one of the preceding embodiments, wherein in step 4) the anti-solvent is added until crystalline particles of the crystalline form of Compound I hydrobromide form.
   Embodiment 148. The method of any one of the preceding embodiments, wherein in step 4) the anti-solvent is selected from the group consisting of ethyl acetate, methyl tert-butyl ether, tetrahydrofuran, and acetone.
   Embodiment 149. The method of any one of the preceding embodiments, wherein in step 4) the anti-solvent is ethyl acetate.
   Embodiment 150. The method of any one of the preceding embodiments, wherein in step 4) ethyl acetate is added over a time period of from about 1 h to about 5 h.
   Embodiment 151. The method of any one of the preceding embodiments, wherein in step 4) ethyl acetate is added over a time period of from about 3 h to about 5 h.
   Embodiment 152. The method of any one of the preceding embodiments, wherein in step 4) ethyl acetate is added over a time period of about 1 h, about 2 h, about 3 h, about 4 h, or about 5 h.
   Embodiment 153. The method of any one of the preceding embodiments, wherein in step 4), the entire amount of ethyl acetate is added at once.
   Embodiment 154. The method of any one of the preceding embodiments, wherein in step 4) ethyl acetate is added in amount of from about 5 volumes to about 15 volumes.
   Embodiment 155. The method of any one of the preceding embodiments, wherein in step 4) ethyl acetate is added in an amount of about 5 volumes, about 6 volumes, about 7 volumes, about 8 volumes, about 9 volumes, about 10 volumes, about 11 volumes, about 12 volumes, about 13 volumes, about 14 volumes, or about 15 volumes.
   Embodiment 156. The method of any one of the preceding embodiments, wherein in step 4) the ethyl acetate is added until crystalline particles of the crystalline form of Compound I hydrobromide form.
   Embodiment 157. The method of any one of the preceding embodiments, wherein in step 4-1) mixture D is stirred for ≥4 h.
   Embodiment 158. The method of any one of the preceding embodiments, wherein in step 4-1) mixture D is stirred for from about 4 h to about 15 h.
   Embodiment 159. The method of any one of the preceding embodiments, wherein in step 4-1) mixture D is stirred for about 4 h, about 5 h, about 6 h, about 7 h, about 8 h, about 9 h, about 10 h, about 11 h, about 12 h, about 13 h, about 14 h, or about 15 h.
   Embodiment 160. The method of any one of the preceding embodiments, wherein in step 5) crude Compound I hydrobromide is isolated from mixture D by filtration.
   Embodiment 161. A crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide):

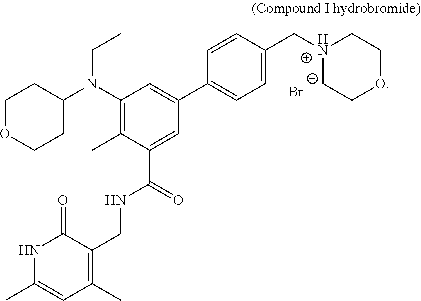

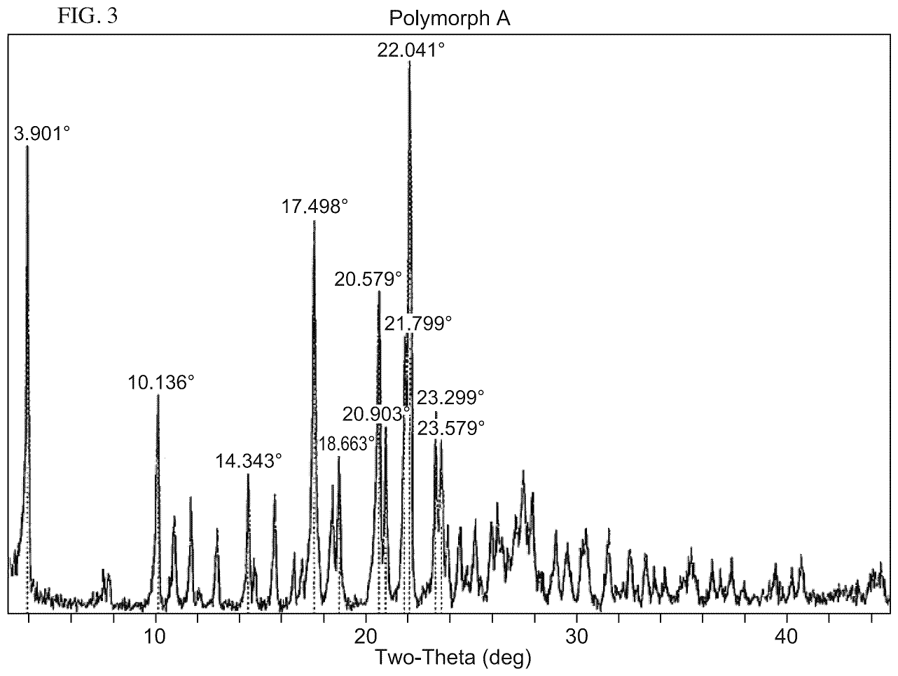

Embodiment 162. A crystalline form of Compound I hydrobromide prepared by a method of any one of the preceding embodiments.
Embodiment 163. The crystalline form of any one of the preceding embodiments, wherein the crystalline form is Polymorph A of Compound I hydrobromide.
Embodiment 164. The crystalline form of any one of the preceding embodiments, wherein the crystalline form exhibits an X-ray powder diffraction pattern having one or two characteristic peaks expressed in 2-theta, selected from the group consisting of 17.5+/−0.3, and 22.0+/−0.3.
Embodiment 165. The crystalline form of any one of the preceding embodiments, wherein the crystalline form exhibits an X-ray powder diffraction pattern having one or more characteristic peaks expressed in degrees 2-theta, selected from the group consisting of 3.9+/−0.3, 17.5+/−0.3, and 22.0+/−0.3.
Embodiment 166. The crystalline form of any one of the preceding embodiments, wherein the crystalline form exhibits an X-ray powder diffraction pattern having characteristic peaks at 3.9+/−0.3, 17.5+/−0.3, and 22.0+/−0.3.
Embodiment 167. The crystalline form of any one of the preceding embodiments, wherein the crystalline form exhibits an X-ray powder diffraction pattern having one or more characteristic peaks expressed in degrees 2-theta, selected from the group consisting of 3.9+/−0.3, 14.3+/−0.3, 18.7+/−0.3, 23.3+/−0.3, and 23.6+/−0.3.
Embodiment 168. The crystalline form of any one of the preceding embodiments, wherein the crystalline form exhibits an X-ray powder diffraction pattern having at least 5 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 169. The crystalline form of any one of the preceding embodiments, wherein the crystalline form exhibits an X-ray powder diffraction pattern having at least 6 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 170. The crystalline form of any one of the preceding embodiments, wherein the crystalline form exhibits an X-ray powder diffraction pattern having at least 7 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 171. The crystalline form of any one of the preceding embodiments, wherein the crystalline form exhibits an X-ray powder diffraction pattern having at least 8 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 172. The crystalline form of any one of the preceding embodiments, wherein the crystalline form exhibits an X-ray powder diffraction pattern having at least 9 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 173. The crystalline form of any one of the preceding embodiments, wherein the crystalline form exhibits an X-ray powder diffraction pattern having at least 10 characteristic peaks expressed in degrees 2-theta, selected from the group consisting of 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 174. The crystalline form of any one of the preceding embodiments, wherein the crystalline form exhibits an X-ray powder diffraction pattern having characteristic peaks expressed in degrees 2-theta, at 3.9+/−0.3, 10.1+/−0.3, 14.3+/−0.3, 17.5+/−0.3, 18.7+/−0.3, 20.6+/−0.3, 20.9+/−0.3, 21.8+/−0.3, 22.0+/−0.3, 23.3+/−0.3 and 23.6+/−0.3.
Embodiment 175. The crystalline form of any one of the preceding embodiments, wherein the crystalline form exhibits an X-ray powder diffraction pattern substantially in accordance with FIG. 3
Embodiment 176. The crystalline form of any one of the preceding embodiments, wherein the crystalline form exhibits an X-ray powder diffraction pattern substantially in accordance with Table 1.
Embodiment 177. The crystalline form of any one of the preceding embodiments, wherein the crystalline form exhibits a differential scanning calorimetry thermogram having a characteristic peak expressed in units of ° C. at a temperature of 255+/−5° C.
Embodiment 178. The crystalline form of any one of the preceding embodiments, wherein the crystalline form exhibits a differential scanning calorimetry thermogram substantially in accordance with FIG. 2.
Embodiment 179. The crystalline form of any one of the preceding embodiments, wherein the crystalline form has a purity of at least 99.8%.
Embodiment 180. The crystalline form of any one of the preceding embodiments, wherein the crystalline form has a purity of 99.8%.
Embodiment 181. The crystalline form of any one of the preceding embodiments, wherein the crystalline form has a purity of 99.9%.
Embodiment 182. The crystalline form of any one of the preceding embodiments, wherein the crystalline form is substantially pure.
Embodiment 183. The crystalline form of any one of the preceding embodiments, wherein the crystalline form of Compound I hydrobromide contains less than 0.2% of derivatives of Compound I.
Embodiment 184. The crystalline form of any one of the preceding embodiments, wherein the crystalline form has a residual ethanol solvent content of 5000 ppm or less.
Embodiment 185. The crystalline form of any one of the preceding embodiments, wherein the crystalline form has a residual ethanol solvent content of 3720 ppm or less.
Embodiment 186. The crystalline form of any one of the preceding embodiments, wherein the crystalline form has a residual ethanol solvent content of 320 ppm or less.
Embodiment 187. The crystalline form of any one of the preceding embodiments, wherein the crystalline form has a residual ethyl acetate solvent content of 5000 ppm or less.
Embodiment 188. The crystalline form of any one of the preceding embodiments, wherein the crystalline form has a residual ethyl acetate solvent content of 2764 ppm or less.
Embodiment 189. The crystalline form of any one of the preceding embodiments, wherein the crystalline form has a residual ethyl acetate solvent content of 75 ppm or less.
Embodiment 190. The crystalline form of any one of the preceding embodiments, wherein the crystalline form has a residual toluene solvent content of 890 ppm or less.
Embodiment 191. The crystalline form of any one of the preceding embodiments, wherein the crystalline form has a residual toluene solvent content of 84 ppm or less.
Embodiment 192. The crystalline form of any one of the preceding embodiments, wherein the crystalline form has a residual toluene solvent content of 20 ppm or less.
Embodiment 193. The crystalline form of any one of the preceding embodiments, wherein the crystalline form of Compound I hydrobromide contains less than 0.2% of N-dealkylation decomposition impurities.
Embodiment 194. The crystalline form of any one of the preceding embodiments, wherein the crystalline form is a polymorph.
Embodiment 195. A polymorph of the crystalline form of any one of the preceding embodiments.
Embodiment 196. The polymorph of any one of the preceding embodiments, wherein the polymorph of Form A is substantially free of other polymorph forms.
Embodiment 197. The polymorph of any one of the preceding embodiments, wherein the polymorph of Form A is free of other polymorph forms.
Embodiment 198. The polymorph of any one of the preceding embodiments, wherein the polymorph contains less than 0.5% of polymorph B.
Embodiment 199. The polymorph of any one of the preceding embodiments, wherein the polymorph is substantially free of polymorph B.
Embodiment 200. The polymorph of any one of the preceding embodiments, wherein the polymorph is free of polymorph B.
Embodiment 201. The polymorph of any one of the preceding embodiments, wherein the polymorph is substantially free of impurities.
Embodiment 202. The polymorph of any one of the preceding embodiments, wherein the polymorph is substantially free of amorphous Compound I.
Embodiment 203. Crystalline particles of a polymorph of any one of the preceding embodiments, wherein the D90 particle size of the particles is from about 15 μm to about 50 μm.
Embodiment 204. A crystalline form of any one of the preceding embodiments, wherein the crystalline form forms particles wherein the D90 particle size of the particles is from about 15 μm to about 50 μm.
Embodiment 205. The crystalline form of any one of the preceding embodiments, wherein the crystalline form forms particles wherein the D90 particle size of the particles is from about 25 μm to about 37 μm, from about 27 μm to about 35 μm, or from about 29 μm to about 33 μm.
Embodiment 206. The crystalline form of any one of the preceding embodiments, wherein the crystalline form forms particles wherein the D90 particle size of the particles is about 25 μm, about 26 μm, about 27 μm, about 28 μm, about 29 μm, about 30 μm, about 31 μm, about 32 μm, about 33 μm, about 34 μm, about 35 μm, about 36 μm, or about 37 am.
Embodiment 207. The crystalline form of any one of the preceding embodiments, wherein the crystalline form forms particles wherein the D90 particle size of the particles is about 31 am.
Embodiment 208. The crystalline form of any one of the preceding embodiments, wherein the crystalline form forms particles wherein at least about 50%, at least about 60%, at least about 70%, at least about 80%, or at least about 90% of the particles have a diameter of from about 6 μm to about 40 μm.
Embodiment 209. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 6 μm to about 40 μm.
Embodiment 210. The crystalline form of any one of the preceding embodiments, wherein the crystalline form forms particles wherein about 100% of the particles have a diameter of from about 6 μm to about 40 μm.
Embodiment 211. The crystalline form of any one of the preceding embodiments, wherein the crystalline form forms particles wherein at least about 50%, at least about 60%, at least about 70%, at least about 80%, or at least about 90% of the particles have a diameter of from about 5 μm to about 50 μm.
Embodiment 212. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 5 μm to about 50 μm.
Embodiment 213. The crystalline form of any one of the preceding embodiments, wherein the crystalline form forms particles wherein about 100% of the particles have a diameter of from about 5 μm to about 50 μm.
Embodiment 214. The crystalline form of any one of the preceding embodiments, wherein the crystalline form forms particles wherein at least about 50%, at least about 60%, at least about 70%, at least about 80%, or at least about 90% of the particles have a diameter of from about 10 μm to about 40 μm.
Embodiment 215. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 10 μm to about 40 μm.
Embodiment 216. The crystalline form of any one of the preceding embodiments, wherein the crystalline form forms particles wherein about 100% of the particles have a diameter of from about 10 μm to about 40 μm.
Embodiment 217. The crystalline form of any one of the preceding embodiments, wherein the crystalline form forms particles wherein at least about 50%, at least about 60%, at least about 70%, at least about 80%, or at least about 90% of the particles have a diameter of from about 15 μm to about 40 μm.
Embodiment 218. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 15 μm to about 40 μm.
Embodiment 219. The crystalline form of any one of the preceding embodiments, wherein the crystalline form forms particles wherein about 100% of the particles have a diameter of from about 15 μm to about 40 μm.
Embodiment 220. The crystalline form of any one of the preceding embodiments, wherein the crystalline form forms particles wherein at least about 50%, at least about 60%, at least about 70%, at least about 80%, or at least about 90% of the particles have a diameter of from about 15 μm to about 35 μm.
Embodiment 221. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 15 μm to about 35 μm.
Embodiment 222. The crystalline form of any one of the preceding embodiments, wherein the crystalline form forms particles wherein about 100% of the particles have a diameter of from about 15 μm to about 35 μm.
Embodiment 223. The crystalline form of any one of the preceding embodiments, wherein the crystalline form forms particles wherein at least about 50%, at least about 60%, at least about 70%, at least about 80%, or at least about 90% of the particles have a diameter of from about 20 μm to about 35 μm.
Embodiment 224. The crystalline form of any one of the preceding embodiments, wherein the crystalline form forms particles wherein at least about 90%, at least about 91%, at least about 92%, at least about 93%, at least about 94%, at least about 95%, at least about 96%, at least about 97%, at least about 98%, or at least about 99% of the particles have a diameter of from about 20 μm to about 35 μm.
Embodiment 225. The crystalline form of any one of the preceding embodiments, wherein the crystalline form forms particles wherein about 100% of the particles have a diameter of from about 20 μm to about 35 μm.
Embodiment 226. The crystalline form of any one of the preceding embodiments, wherein the crystalline form forms particles wherein at least about 60% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 227. The crystalline form of any one of the preceding embodiments, wherein the crystalline form forms particles wherein at least about 70% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 228. The crystalline form of any one of the preceding embodiments, wherein the crystalline form forms particles wherein at least about 80% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 229. The crystalline form of any one of the preceding embodiments, wherein the crystalline form forms particles wherein at least about 90% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 230. The crystalline form of any one of the preceding embodiments, wherein the crystalline form forms particles wherein at least about 95% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 231. The crystalline form of any one of the preceding embodiments, wherein the crystalline form forms particles wherein about 100% of the particles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 232. The crystalline form of any one of the preceding embodiments, wherein the crystalline form forms particles wherein the particles have a particle size distribution with a relative span of from about 1 to about 5, or from about 2 to about 4.
Embodiment 233. The crystalline form of any one of the preceding embodiments, wherein the crystalline form forms particles wherein the particles have a particle size distribution with a relative span of about 1.0, about 1.1, about 1.2, about 1.3, about 1.4, about 1.5, about 1.6, about 1.7, about 1.8, about 1.9, or about 2.0.
Embodiment 234. The crystalline form of any one of the preceding embodiments, wherein the crystalline form forms particles wherein the particles have a particle size distribution with a relative span of about 2.5, about 2.7, or about 3.0.
Embodiment 235. A pharmaceutical composition comprising the crystalline form of any one of the preceding embodiments and one or more pharmaceutically acceptable excipients.
Embodiment 236. A plurality of microparticles of a crystalline form of any one of the preceding embodiments.
Embodiment 237. A plurality of microparticles of Compound I hydrobromide, wherein the microparticles are crystalline microparticles.
Embodiment 238. A plurality of microparticles of a crystalline form of Compound I hydrobromide, wherein the crystalline form is prepared by a method of any one of the preceding embodiments.
Embodiment 239. The plurality of microparticles of any one of the preceding embodiments, wherein the D90 particle size of the microparticles is from about 15 μm to about 50 μm.
Embodiment 240. The plurality of microparticles of any one of the preceding embodiments, wherein the D90 particle size of the microparticles is from about 25 μm to about 37 μm, from about 27 μm to about 35 μm, or from about 29 μm to about 33 μm.
Embodiment 241. The plurality of microparticles of any one of the preceding embodiments, wherein the D90 particle size of the microparticles is about 25 μm, about 26 μm, about 27 μm, about 28 μm, about 29 μm, about 30 μm, about 31 μm, about 32 μm, about 33 μm, about 34 μm, about 35 μm, about 36 μm, or about 37 am.
Embodiment 242. The plurality of microparticles of any one of the preceding embodiments, wherein the D90 particle size of the microparticles is about 31 am.
Embodiment 243. The plurality of microparticles of any one of the preceding embodiments, wherein at least about 50%, at least about 60%, at least about 70%, at least about 80%, or at least about 90% of the microparticles have a diameter of from about 6 μm to about 40 μm.
Embodiment 244. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 6 μm to about 40 μm.
Embodiment 245. The plurality of microparticles of any one of the preceding embodiments, wherein about 100% of the microparticles have a diameter of from about 6 μm to about 40 μm.
Embodiment 246. The plurality of microparticles of any one of the preceding embodiments, wherein at least about 50%, at least about 60%, at least about 70%, at least about 80%, or at least about 90% of the microparticles have a diameter of from about 5 μm to about 50 μm.
Embodiment 247. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 5 μm to about 50 μm.
Embodiment 248. The plurality of microparticles of any one of the preceding embodiments, wherein about 100% of the microparticles have a diameter of from about 5 μm to about 50 μm.
Embodiment 249. The plurality of microparticles of any one of the preceding embodiments, wherein at least about 50%, at least about 60%, at least about 70%, at least about 80%, or at least about 90% of the microparticles have a diameter of from about 10 μm to about 40 μm.
Embodiment 250. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 10 μm to about 40 μm.
Embodiment 251. The plurality of microparticles of any one of the preceding embodiments, wherein about 100% of the microparticles have a diameter of from about 10 μm to about 40 μm.
Embodiment 252. The plurality of microparticles of any one of the preceding embodiments, wherein at least about 50%, at least about 60%, at least about 70%, at least about 80%, or at least about 90% of the microparticles have a diameter of from about 15 μm to about 40 μm.
Embodiment 253. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 15 μm to about 40 μm.
Embodiment 254. The plurality of microparticles of any one of the preceding embodiments, wherein about 100% of the microparticles have a diameter of from about 15 μm to about 40 μm.
Embodiment 255. The plurality of microparticles of any one of the preceding embodiments, wherein at least about 50%, at least about 60%, at least about 70%, at least about 80%, or at least about 90% of the microparticles have a diameter of from about 15 μm to about 35 μm.
Embodiment 256. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 15 μm to about 35 μm.
Embodiment 257. The plurality of microparticles of any one of the preceding embodiments, wherein about 100% of the microparticles have a diameter of from about 15 μm to about 35 μm.
Embodiment 258. The plurality of microparticles of any one of the preceding embodiments, wherein at least about 50%, at least about 60%, at least about 70%, at least about 80%, or at least about 90% of the microparticles have a diameter of from about 20 μm to about 35 μm.
Embodiment 259. The plurality of microparticles of any one of the preceding embodiments, wherein at least about 90%, at least about 91%, at least about 92%, at least about 93%, at least about 94%, at least about 95%, at least about 96%, at least about 97%, at least about 98%, or at least about 99% of the microparticles have a diameter of from about 20 μm to about 35 μm.
Embodiment 260. The plurality of microparticles of any one of the preceding embodiments, wherein about 100% of the microparticles have a diameter of from about 20 μm to about 35 μm.
Embodiment 261. The plurality of microparticles of any one of the preceding embodiments, wherein at least about 60% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 262. The plurality of microparticles of any one of the preceding embodiments, wherein at least about 70% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 263. The plurality of microparticles of any one of the preceding embodiments, wherein at least about 80% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 264. The plurality of microparticles of any one of the preceding embodiments, wherein at least about 90% of the microparticles have a diameter of about 20 μm, about 21 μm, about 22 μm, about 23 μm, about 24 μm, about 25 μm, about 26 μm, about 27 μm, about 28 μm, about 29 μm, about 31 μm, about 31 μm, about 32 μm, about 33 μm, about 34 μm, or about 35 μm.
Embodiment 265. The plurality of microparticles of any one of the preceding embodiments, wherein at least about 95% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 266. The plurality of microparticles of any one of the preceding embodiments, wherein about 100% of the microparticles have a diameter of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, or about 35 μm.
Embodiment 267. The plurality of microparticles of any one of the preceding embodiments, wherein the plurality of microparticles has a particle size distribution with a relative span of from about 1 to about 5, or from about 2 to about 4.
Embodiment 268. The plurality of microparticles of any one of the preceding embodiments, wherein the plurality of microparticles has a particle size distribution with a relative span of about 1.0, about 1.1, about 1.2, about 1.3, about 1.4, about 1.5, about 1.6, about 1.7, about 1.8, about 1.9, or about 2.0.
Embodiment 269. The plurality of microparticles of any one of the preceding embodiments, wherein the plurality of microparticles has a particle size distribution with a relative span of about 2.5, about 2.7, or about 3.0.
Embodiment 270. The plurality of microparticles of any one of the preceding embodiments, wherein the microparticles are crystalline particles.
Embodiment 271. A pharmaceutical composition comprising the plurality of microparticles of any one of the preceding embodiments and one or more pharmaceutically acceptable excipients.
Embodiment 272. A method of treating cancer, comprising administering to a subject in need thereof a polymorph of any one of the preceding embodiments.
Embodiment 273. The polymorph of any one of the preceding embodiments for use in the treatment of cancer.
Embodiment 274. The polymorph of any one of the preceding embodiments for use in the manufacture of a medicament for treating cancer.
Embodiment 275. Use of the polymorph of any one of the preceding embodiments in the manufacture of a medicament for the treatment of cancer.
Embodiment 276. A method of treating cancer, comprising administering to a subject in need thereof crystalline particles of a polymorph of any one of the preceding embodiments, wherein the D90 particle size of the particles is from about 15 μm to about 50 μm.
Embodiment 277. Crystalline particles of a polymorph of any one of the preceding embodiments, wherein the D90 particle size of the particles is from about 15 μm to about 50 μm for use in the treatment of cancer.
Embodiment 278. Crystalline particles of a polymorph of any one of the preceding embodiments, wherein the D90 particle size of the particles is from about 15 μm to about 50 μm for use in the manufacture of a medicament for treating cancer.
Embodiment 279. Use of crystalline particles of a polymorph of any one of the preceding embodiments, wherein the D90 particle size of the particles is from about 15 μm to about 50 μm in the manufacture of a medicament for the treatment of cancer.
Embodiment 280. A pharmaceutical composition comprising crystalline particles comprising a polymorph of Compound I hydrobromide and one or more pharmaceutically acceptable excipients, wherein the polymorph is prepared by a method of any one of the preceding embodiments.
Embodiment 281. A pharmaceutical composition comprising crystalline particles comprising a polymorph of Compound I hydrobromide and one or more pharmaceutically acceptable excipients, wherein the D90 particle size of the particles is from about 15 μm to about 50 μm, and wherein the polymorph is prepared by a method of any one of the preceding embodiments.
Embodiment 282. A pharmaceutical composition comprising a polymorph of any one of the preceding embodiments.
Embodiment 283. A pharmaceutical composition comprising crystalline particles of a polymorph of any one of the preceding embodiments, and one or more pharmaceutically acceptable excipients wherein the D90 particle size of the particles is from about 15 μm to about 50 μm.
Embodiment 284. The pharmaceutical composition of any one of the preceding embodiments, wherein the one or more pharmaceutically acceptable excipients are selected from low-substituted hydroxypropylcellulose, hydroxypropyl cellulose, and a combination thereof.
Embodiment 285. The pharmaceutical composition of any one of the preceding embodiments, wherein the concentration of Compound I hydrobromide in the composition is from about 50 wt. % to about 60 wt. %.
Embodiment 286. The pharmaceutical composition of any one of the preceding embodiments, wherein the concentration of Compound I hydrobromide in the composition is about 57 wt. %.
Embodiment 287. The pharmaceutical composition of any one of the preceding embodiments, wherein the concentration of Compound I hydrobromide in the composition is 57.1 wt. %.
Embodiment 288. The pharmaceutical composition of any one of the preceding embodiments, wherein the composition further comprises lactose monohydrate, sodium starch glycolate, or magnesium stearate, or a combination thereof.
Embodiment 289. The pharmaceutical composition of any one of the preceding embodiments, wherein the composition comprises from about 10 wt. % to about 20 wt. % lactose monohydrate.
Embodiment 290. The pharmaceutical composition of any one of the preceding embodiments, wherein the composition comprises about 17 wt. % lactose monohydrate.
Embodiment 291. The pharmaceutical composition of any one of the preceding embodiments, wherein the composition comprises about 15 wt. % to about 25 wt. % low-substituted hydroxypropyl cellulose, sodium starch glycolate, or a combination thereof.
Embodiment 292. The pharmaceutical composition of any one of the preceding embodiments, wherein the composition comprises about 15 wt. % low-substituted hydroxypropyl cellulose.
Embodiment 293. The pharmaceutical composition of any one of the preceding embodiments, wherein the composition comprises about 5 wt. % sodium starch glycolate.
Embodiment 294. The pharmaceutical composition of any one of the preceding embodiments, wherein the composition comprises about 1 wt. % to about 10 wt. % hydroxypropyl cellulose.
Embodiment 295. The pharmaceutical composition of any one of the preceding embodiments, wherein the composition comprises about 4 wt. % hydroxypropyl cellulose.
Embodiment 296. The pharmaceutical composition of any one of the preceding embodiments, wherein the composition comprises about 0.5 wt. % to about 5 wt. % magnesium stearate.
Embodiment 297. The pharmaceutical composition of any one of the preceding embodiments, wherein the composition comprises about 2 wt. % magnesium stearate.
Embodiment 298. The pharmaceutical composition of any one of the preceding embodiments, wherein the coating composition is present in an amount of about 1-10 wt. %.
Embodiment 299. The pharmaceutical composition of any one of the preceding embodiments, wherein the one or more pharmaceutical excipients comprise lactose monohydrate; low-substituted hydroxypropyl cellulose; hydroxypropyl cellulose; sodium starch glycolate; and magnesium stearate.
Embodiment 300. The pharmaceutical composition of any one of the preceding embodiments, wherein the composition comprises the crystalline form of Compound I hydrobromide in an amount of about 50 wt. % to about 60 wt. %, lactose monohydrate in an amount of about 10-20 wt. %; low-substituted hydroxypropyl cellulose in an amount of about 11-19 wt. %; sodium starch glycolate in an amount of about 3-7 wt. %; hydroxypropyl cellulose in an amount of about 1-10 wt. %; and magnesium stearate in an amount of about 0.5-5 wt. %.
Embodiment 301. The pharmaceutical composition of any one of the preceding embodiments, wherein the composition comprises the crystalline form of Compound I hydrobromide in an amount of about 57 wt. %; lactose monohydrate in an amount of about 17 wt. %; low-substituted hydroxypropyl cellulose in an amount of about 15 wt. %; sodium starch glycolate in an amount of about 5 wt. %; hydroxypropyl cellulose in an amount of about 4 wt. %; and magnesium stearate in an amount of about 2 wt. %.
Embodiment 302. The pharmaceutical composition of any one of the preceding embodiments, wherein the composition comprises the crystalline form of Compound I hydrobromide in an amount of about 55 wt. %; lactose monohydrate in an amount of about 17 wt. %; low-substituted hydroxypropyl cellulose in an amount of about 15 wt. %; sodium starch glycolate in an amount of about 5 wt. %; hydroxypropyl cellulose in an amount of about 4 wt. %; and magnesium stearate in an amount of about 2 wt. %.
Embodiment 303. The pharmaceutical composition of any one of the preceding embodiments, further comprising a coating composition.
Embodiment 304. The pharmaceutical composition of any one of the preceding embodiments, further comprising a coating composition in an amount of about 4 wt. %.
Embodiment 305. The pharmaceutical composition of any one of the preceding embodiments, wherein the composition consists of the crystalline form of Compound I hydrobromide in an amount of about 57 wt. %; lactose monohydrate in an amount of about 17 wt. %; low-substituted hydroxypropyl cellulose in an amount of about 15 wt. %; sodium starch glycolate in an amount of about 5 wt. %; hydroxypropyl cellulose in an amount of about 4 wt. %; and magnesium stearate in an amount of about 2 wt. %.
Embodiment 306. The pharmaceutical composition of any one of the preceding embodiments, wherein the composition consists of the crystalline form of Compound I hydrobromide in an amount of about 55 wt. %; lactose monohydrate in an amount of about 17 wt. %; low-substituted hydroxypropyl cellulose in an amount of about 15 wt. %; sodium starch glycolate in an amount of about 5 wt. %; hydroxypropyl cellulose in an amount of about 4 wt. %; magnesium stearate in an amount of about 2 wt. % and a coating composition in an amount of about 4 wt. %.
Embodiment 307. The pharmaceutical composition of any one of the preceding embodiments, wherein the composition comprises the crystalline form of Compound I hydrobromide in an amount of about 50 wt. % to about 60 wt. %, lactose monohydrate in an amount of about 10-20 wt. %; low-substituted hydroxypropyl cellulose in an amount of about 11-19 wt. %; sodium starch glycolate in an amount of about 3-7 wt. %; hydroxypropyl cellulose in an amount of about 1-10 wt. %; and magnesium stearate in an amount of about 0.5-5 wt. % and a coating composition in an amount of about 1-10 wt. %.
Embodiment 308. The pharmaceutical composition of any one of the preceding embodiments, wherein the coating composition is a hydroxypropyl methylcellulose-based film coating.
Embodiment 309. The pharmaceutical composition of any one of the preceding embodiments, wherein the coating composition comprises hydroxypropyl methylcellulose.
Embodiment 310. The pharmaceutical formulation of any one of the preceding embodiments, wherein the coating composition comprises talc.
Embodiment 311. The pharmaceutical formulation of any one of the preceding embodiments, wherein the coating composition comprises macrogol.
Embodiment 312. The pharmaceutical formulation of any one of the preceding embodiments, wherein the coating composition comprises titanium dioxide.
Embodiment 313. The pharmaceutical formulation of any one of the preceding embodiments, wherein the coating composition comprises iron (III) oxide.
Embodiment 314. The pharmaceutical formulation of any one of the preceding embodiments, wherein the coating composition comprises iron(III) oxide-hydroxide.
Embodiment 315. The pharmaceutical composition of any of the preceding embodiments, wherein the coating composition is an Opadry® film coating.
Embodiment 316. The pharmaceutical composition of any one of the preceding embodiments, wherein the coating composition is Opadry® 03F45063 RED.
Embodiment 317. The pharmaceutical composition of any one of the preceding embodiments, wherein the coating composition is Opadry® 03F220119 YELLOW.
Embodiment 318. The pharmaceutical composition of any one of the preceding embodiments, wherein the composition is in the form of a tablet.
Embodiment 319. The pharmaceutical composition of any one of the preceding embodiments, wherein the composition is in the form of a tablet and wherein the tablet comprises the crystalline form of Compound I hydrobromide in an amount of about 28.5 mg, about 57 mg, about 114 mg, about 228, or about 456 mg.
Embodiment 320. A method of treating cancer, comprising administering to a subject in need thereof a pharmaceutical composition of any one of the preceding embodiments.
Embodiment 321. The pharmaceutical composition of any one of the preceding embodiments for use in the treatment of cancer.
Embodiment 322. The pharmaceutical composition of any one of the preceding embodiments for use in the manufacture of a medicament for treating cancer.
Embodiment 323. Use of the pharmaceutical composition of any one of the preceding embodiments in the manufacture of a medicament for the treatment of cancer.

### EQUIVALENTS

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly indicates otherwise.

We claim:

1. A plurality of microparticles of a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide): 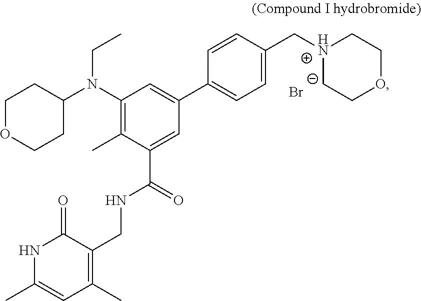
wherein at least about 60% of the microparticles have a diameter of from about 5 μm to about 50 μm.

2. (canceled)

3. The plurality of microparticles of claim 1, wherein at least about 80% of the microparticles have a diameter of from about 5 μm to about 50 μm.

4. The plurality of microparticles of claim 1, wherein at least about 90% of the microparticles have a diameter of from about 5 μm to about 50 μm.

5-6. (canceled)

7. The plurality of microparticles of claim 1, wherein at least about 80% of the microparticles have a diameter of from about 6 μm to about 40 μm.

8. The plurality of microparticles of claim 1, wherein at least about 90% of the microparticles have a diameter of from about 6 μm to about 40 μm.

9-10. (canceled)

11. The plurality of microparticles of claim 1, wherein at least about 80% of the microparticles have a diameter of from about 15 μm to about 40 μm.

12. The plurality of microparticles of claim 1, wherein at least about 90% of the microparticles have a diameter of from about 15 μm to about 40 μm.

13. The plurality of microparticles of claim 1, wherein the crystalline form is Polymorph A of Compound I hydrobromide, wherein the Polymorph A exhibits an X-ray powder diffraction pattern having one or two characteristic peaks expressed in 2-theta selected from the group consisting of 17.5+/−0.3 and 22.0+/−0.3, wherein the crystalline form is substantially free of other polymorph forms.

14. (canceled)

15. The plurality of microparticles of claim 1, wherein the microparticles are prepared by a method comprising:
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), ethanol, and toluene to form mixture A; after step 1):
step 2) adding hydrobromic acid to mixture A to form a mixture B, wherein Compound I hydrobromide is formed; after step 2):
step a) mixing Compound I hydrobromide, ethanol, and water, wherein the vol/vol ratio of ethanol:water is from about 92:8 to about 87:13, to form a first mixture; and
after step a):
step b) adding a seed to the first mixture to form a second mixture.

16. A plurality of microparticles of a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide): 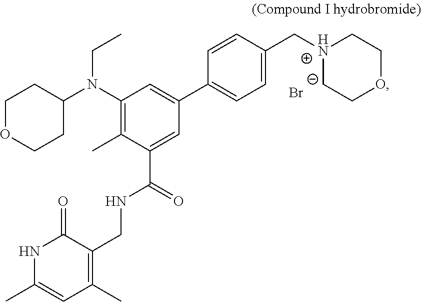
wherein the D90 particle size of the microparticles is from about 15 μm to about 50 μm.

17. The plurality of microparticles of claim 16, wherein the crystalline form is Polymorph A of Compound I hydrobromide, wherein the Polymorph A exhibits an X-ray powder diffraction pattern having one or two characteristic peaks expressed in 2-theta selected from the group consisting of 17.5+/−0.3 and 22.0+/−0.3.

18. The plurality of microparticles of claim 17, wherein the crystalline form is substantially free of other polymorph forms.

19. The plurality of microparticles of claim 16, wherein the microparticles are prepared by a method comprising:
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), ethanol, and toluene to form mixture A; after step 1):
step 2) adding hydrobromic acid to mixture A to form a mixture B, wherein Compound I hydrobromide is formed; after step 2):
step a) mixing Compound I hydrobromide, ethanol, and water, wherein the vol/vol ratio of ethanol:water is from about 92:8 to about 87:13, to form a first mixture; and after step a):
step b) adding a seed to the first mixture to form a second mixture.

20. A plurality of microparticles of a crystalline form of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide hydrobromide (Compound I hydrobromide): 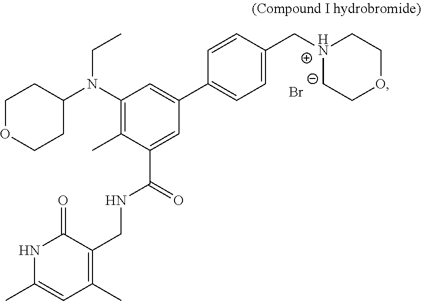
wherein
(i) at least about 60% of the microparticles have a diameter of from about 5 μm to about 50 μm;
(ii) the D90 particle size of the microparticles is from about 15 μm to about 50 μm; and
(iii) the crystalline form is Polymorph A of Compound I hydrobromide, wherein the Polymorph A exhibits an X-ray powder diffraction pattern having one or two characteristic peaks expressed in 2-theta selected from the group consisting of 17.5+/−0.3 and 22.0+/−0.3.

21. (canceled)

22. The plurality of microparticles of claim 20, wherein at least about 80% of the microparticles have a diameter of from about 6 μm to about 40 μm.

23. The plurality of microparticles of claim 20, wherein at least about 90% of the microparticles have a diameter of from about 6 μm to about 40 μm.

24. The plurality of microparticles of claim 20, wherein the microparticles are prepared by a method comprising:
step 1) mixing N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (Compound I), ethanol, and toluene to form mixture A; after step 1):
step 2) adding hydrobromic acid to mixture A to form a mixture B, wherein Compound I hydrobromide is formed; after step 2):
step a) mixing Compound I hydrobromide, ethanol, and water, wherein the vol/vol ratio of ethanol:water is from about 92:8 to about 87:13, to form a first mixture; and after step a):
step b) adding a seed to the first mixture to form a second mixture.

25. A pharmaceutical composition comprising a plurality of microparticles of claim 1.

26. A pharmaceutical composition comprising a plurality of microparticles of claim 16.

27. A pharmaceutical composition comprising a plurality of microparticles of claim 20.

28. The pharmaceutical composition of claim 25, further comprising:
lactose monohydrate in an amount of from about 10 wt. % to about 20 wt. %;
low-substituted hydroxypropyl cellulose in an amount of from about 11 wt. % to about 19 wt. %;
sodium starch glycolate in an amount of from about 3 wt. % to about 7 wt. %;
hydroxypropyl cellulose in an amount of from about 1 wt. % to about 10 wt. %;
magnesium stearate in an amount of from about 0.5 wt. % to about 5 wt. %; and
a coating composition in an amount of from about 1 wt. % to about 10 wt. %.

29. The pharmaceutical composition of claim 26, further comprising:
lactose monohydrate in an amount of from about 10 wt. % to about 20 wt. %;
low-substituted hydroxypropyl cellulose in an amount of from about 11 wt. % to about 19 wt. %;
sodium starch glycolate in an amount of from about 3 wt. % to about 7 wt. %;
hydroxypropyl cellulose in an amount of from about 1 wt. % to about 10 wt. %;
magnesium stearate in an amount of from about 0.5 wt. % to about 5 wt. %; and
a coating composition in an amount of from about 1 wt. % to about 10 wt. %.

30. The pharmaceutical composition of claim 27, further comprising:
lactose monohydrate in an amount of from about 10 wt. % to about 20 wt. %;
low-substituted hydroxypropyl cellulose in an amount of from about 11 wt. % to about 19 wt. %;
sodium starch glycolate in an amount of from about 3 wt. % to about 7 wt. %;
hydroxypropyl cellulose in an amount of from about 1 wt. % to about 10 wt. %;
magnesium stearate in an amount of from about 0.5 wt. % to about 5 wt. %; and
a coating composition in an amount of from about 1 wt. % to about 10 wt. %.

31. The plurality of microparticles of claim 1, wherein the crystalline form of Compound I hydrobromide has a residual ethanol solvent content of 5000 ppm or less, a residual ethyl acetate solvent content of 5000 ppm or less, and a residual toluene solvent content of 890 ppm or less.

32. The plurality of microparticles of claim 1, wherein the crystalline form of Compound I hydrobromide contains less than 0.2% of N dealkylation decomposition impurities.
